(19) United States
(12) Patent Application Publication
(10) Pub. No.: US 2025/0034252 A1
(43) Pub. Date: Jan. 30, 2025

(54) MANABODIES TARGETING P53 TUMOR ANTIGENS AND METHODS OF USING

(71) Applicant: The Johns Hopkins University, Baltimore, MD (US)

(72) Inventors: Bert Vogelstein, Baltimore, MD (US); Kenneth W. Kinzler, Frankford, DE (US); Nickolas Papadopoulos, Towson, MD (US); Shibin Zhou, Owings Mills, MD (US); Sarah DiNapoli, Baltimore, MD (US); Emily Han-Chung Hsiue, Baltimore, MD (US); Brian J. Mog, Baltimore, MD (US); Katharine M. Wright, Baltimore, MD (US); Sandra B. Gabelli, Ellicott City, MD (US)

(21) Appl. No.: 18/715,847

(22) PCT Filed: Dec. 15, 2022
(86) PCT No.: PCT/US2022/053065

§ 371 Date: Jun. 3, 2024

Related U.S. Application Data
(60) Provisional application No. 63/290,353, filed on Dec. 16, 2021.

(51) Int. Cl. C07K 16/28; A61K 39/00; A61P 35/00; C07K 16/30

(52) U.S. Cl. CPC C07K 16/2809; A61P 35/00; C07K 16/30; A61K 2039/505; C07K 2317/31; C07K 2317/565; C07K 2317/622; C07K 2317/626; C07K 2317/92

(57) ABSTRACT

Described herein are methods and compositions for assessing a mammal having or suspected of having cancer and/or for treating a mammal having cancer. For example, molecules including one or more antigen-binding domains (e.g., a single-chain variable fragment (scFv)) that can bind to a modified peptide (e.g., a tumor antigen), as well as method for using such molecules, are provided.

### CROSS-REFERENCE TO RELATED APPLICATIONS

This application claims priority to U.S. Provisional Patent Application No. 63/290,353, filed on Dec. 16, 2021, which is incorporated herein by reference in its entirety.

### FEDERALLY SPONSORED RESEARCH OR DEVELOPMENT

This invention was made with government support under grant CA006973 awarded by the National Institutes of Health. The government has certain rights in the invention.

### SEQUENCE LISTING

This application contains a Sequence Listing that has been submitted electronically as an XML file named “44807-0407W01_SL_ST26.XML.” The XML file, created on Dec. 8, 2022, is 82,067 bytes in size. The material in the XML file is hereby incorporated by reference in its entirety.

### TECHNICAL FIELD

Described herein are methods and compositions for assessing a mammal having or suspected of having cancer and/or for treating a mammal having cancer. For example, molecules including one or more antigen-binding domains (e.g., a single-chain variable fragment (scFv)) that can bind to a modified peptide (e.g., a tumor antigen), as well as method for using such molecules, are provided.

### BACKGROUND

In recent years, antibody and cell-based immunotherapies have emerged as promising cancer treatments. However, many immunotherapies targeting antigens that have elevated expression on tumor cells have significant toxicities due to low expression of the same target on normal tissues. An ideal immunotherapeutic would be able to specifically identify cancer cells harboring essential driver mutations while leaving normal cells untouched. Targeting cells containing these mutations is often difficult as many common mutations are in driver genes that encode intracellular proteins that are not directly accessible to immunotherapies.

Somatic mutations in cancer are ideal targets for cancer therapy as they are expressed only in tumor cells and not in normal cells. Targeting driver gene proteins (broadly subdivided into oncogene proteins and tumor suppressor proteins) have added benefits. First, these mutations typically occur early during the development of the tumor, thus essentially all daughter cancer cells will contain the mutation. Second, the tumor's dependence on their oncogenic-endowing capacity makes resistance less likely. Finally, driver gene proteins tend to have hotspot mutations shared among many patients, thus a therapy targeting a single mutation could be applied to a broad patient population.

Most mutant proteins, including most mutant driver gene proteins, are intracellular. While small molecules can target intracellular proteins, developing small molecules that can specifically inhibit the activity of a mutant driver gene and not its wild-type (WT) counterpart has remained out of reach for the majority of such driver gene proteins. Antibodies, which can have the capacity to distinguish a single amino acid mutation, can typically only target extracellular epitopes.

The immune system samples the intracellular contents of cells through antigen processing and presentation. Following protein proteolysis, a fraction of the resulting peptides are loaded onto a human leukocyte antigen (HLA) and sent to the cell surface where they serve as a way for T cells, via their T cell receptor (TCR), to distinguish self from non-self peptides. For example, a virally-infected cell will present viral peptides in its HLA, triggering T cells to kill that cell. Similarly, in cancer, mutant peptides can be presented in an HLA on the cancer cell surface, referred to as MANAs, for Mutation-Associated Neo-Antigens. In some cases, and to varying degrees, patients may mount an anti-cancer T cell response against these mutant-peptide-HLA neoantigens, and checkpoint blockade antibodies can further augment this response. However, many patients, particularly those with a low mutational burden, cannot mount a sufficient anti-cancer T cell response. A therapy or diagnostic specifically targeting MANAs could therefore provide a truly tumor-specific method to diagnose or treat cancer.

HLA class I proteins are present on all nucleated cells. There are three classical HLA class I genes, A, B, and C, each of which are highly polymorphic. Each HLA allele has a particular peptide-binding motif, and as a result, only certain peptides will bind to certain HLA alleles.

There is a continuing need in the art to develop new and improved methods to diagnose, monitor, and effectively treat cancers.

### SUMMARY

Identification of therapeutic targets highly specific to cancer cells is one of the greatest challenges for developing an effective cancer therapy.

Described herein are methods and compositions for treating a mammal having cancer. For example, this document provides methods and materials for using one or more molecules including one or more antigen-binding domains (e.g., scFvs) that can bind to a modified peptide (e.g., a modified peptide present in a peptide-HLA-beta-2 microglobulin (b2M or 02M) complex) to treat a mammal having a cancer (e.g., a cancer expressing the modified peptide). In some cases, one or more molecules including one or more antigen-binding domains (e.g., scFvs) that can bind to a modified peptide (e.g., a modified peptide present in a peptide-HLA-02M complex) can be administered to a mammal having a cancer (e.g., a cancer expressing the modified peptide) to treat the mammal.

As demonstrated herein, scFvs were identified that target (e.g., bind to) numerous MANAs present in HLA-restricted MANAs derived from the common cancer driver mutation p53 R175H (“R175H”). Also as demonstrated herein, the scFvs were used to design bispecific antibodies capable of inducing MANA-dependent T cell activation that can lead to recognition and killing of cells (e.g., cancer cells) expressing MANAs.

MANAs can be used as highly specific cancer targets because they are not present in normal tissue(s). The ability to specifically target MANAs provides a tumor-specific method to diagnose and/or treat cancer. For example, scFvs specifically targeting MANAs can be used in full-length antibodies or fragments thereof, antibody drug conjugates (ADCs), antibody radionuclide conjugates, T cells expressing a chimeric antigen receptor (CARTs), or bispecific antibodies to diagnose and/or treat a mammal having cancer. Further, an antibody that can bind to a MANA (a MANAbody), or a fragment thereof capable of binding to a MANA, have the potential of becoming widely applicable and genetically predictable off-the-shelf targeted cancer immunotherapy.

In recent years, antibody and cell-based immunotherapies have emerged as promising cancer treatments. However, many immunotherapies targeting antigens that have elevated expression on tumor cells have significant toxicities due to low expression of the same target on normal tissues. An ideal immunotherapeutic would be able to specifically identify cancer cells harboring essential driver mutations while leaving normal cells untouched. Targeting cells containing these mutations is often difficult as many common mutations are in driver genes that encode intracellular proteins that are not directly accessible to immunotherapies. The potential for targeting cancer-specific mutations presented as mutant peptides bound to cells' major histocompatibility complexes (pMHCs) has been demonstrated. Using bispecific antibodies or chimeric antigen receptor (CAR) T cells to target cells presenting these mutant peptides offers a genetically specific therapy that identifies cells containing mutant proteins that would otherwise be hidden from immune surveillance.

Yet, a need remains for improved antibodies with high affinity for these mutant peptides.

Thus, described herein are molecules comprising a first antigen-binding domain comprising: (i) an scFv light chain CDR1 comprising or consisting of SEQ ID NO: 8; (ii) an scFv light chain CDR2 comprising or consisting of SEQ ID NO: 9 or SEQ ID NO: 23; (iii) an scFv light chain CDR3 comprising or consisting of SEQ ID NO: 10; (iv) an scFV heavy chain CDR1 comprising or consisting of SEQ ID NO: 17; (v) an scFV heavy chain CDR2 comprising or consisting of SEQ ID NO: 18 or SEQ ID NO: 27; and (vi) an scFV heavy chain CDR3 comprising or consisting of SEQ ID NO: 19.

In some embodiments, the first antigen-binding domain comprises: (a) the scFv light chain CDR2 comprising or consisting of SEQ ID NO: 23; or (b) the scFv heavy chain CDR2 comprising or consisting of SEQ ID NO: 27; or (c) the scFv light chain CDR2 comprising or consisting of SEQ ID NO: 23 and the scFv heavy chain CDR2 comprising or consisting of SEQ ID NO: 27.

In some embodiments, the first antigen-binding domain comprises: (i) an scFv light chain comprising a sequence at least 90% identical to SEQ ID NO: 7 or SEQ ID NO: 22; and (ii) an scFv heavy chain comprising a sequence at least 90% identical to SEQ ID NO: 16 or SEQ ID NO: 26. In some embodiments, the first antigen-binding domain comprises: (a) the scFv light chain comprising the sequence at least 90% identical to SEQ ID NO: 7 and the scFv heavy chain comprising a sequence at least 90% identical to SEQ ID NO: 26; or (b) the scFv light chain comprising a sequence at least 90% identical to SEQ ID NO: 22 and the scFv heavy chain comprising a sequence at least 90% identical to SEQ ID NO: 16; or (c) the scFv light chain comprising a sequence at least 90% identical to SEQ ID NO: 22 and the scFv heavy chain comprising a sequence at least 90% identical to SEQ ID NO: 26.

In some embodiments, the first antigen-binding domain comprises: (i) an scFv light chain comprising a sequence at least 98% identical to SEQ ID NO: 7 or SEQ ID NO: 22; and (ii) an scFv heavy chain comprising a sequence at least 98% identical to SEQ ID NO: 16 or SEQ ID NO: 26. In some embodiments, the first antigen-binding domain comprises: (a) the scFv light chain comprising the sequence at least 98% identical to SEQ ID NO: 7 and the scFv heavy chain comprising a sequence at least 98% identical to SEQ ID NO: 26; or (b) the scFv light chain comprising a sequence at least 98% identical to SEQ ID NO: 22 and the scFv heavy chain comprising a sequence at least 98% identical to SEQ ID NO: 16; or (c) the scFv light chain comprising a sequence at least 98% identical to SEQ ID NO: 22 and the scFv heavy chain comprising a sequence at least 98% identical to SEQ ID NO: 26.

In some embodiments, the first antigen-binding domain comprises: (i) an scFv light chain comprising or consisting of SEQ ID NO: 7 or SEQ ID NO: 22; and (ii) an scFv heavy chain comprising or consisting of SEQ ID NO: 16 or SEQ ID NO: 26. In some embodiments, the first antigen-binding domain comprises: (a) the scFv light chain comprising or consisting of SEQ ID NO: 7 and the scFv heavy chain comprising or consisting of SEQ ID NO: 26; or (b) the scFv light chain comprising or consisting of SEQ ID NO: 22 and the scFv heavy chain comprising or consisting of SEQ ID NO: 16; or (c) the scFv light chain comprising or consisting of SEQ ID NO: 22 and the scFv heavy chain comprising or consisting of SEQ ID NO: 26.

In some embodiments, the molecule is selected from the group consisting of an antibody, an antibody fragment, a single chain variable fragment (scFv), a chimeric antigen receptor (CAR), a T cell receptor (TCR), a TCR mimic, a tandem scFv, a bispecific T cell engager, a diabody, a single-chain diabody (scDb), an scFv-Fc, a bispecific antibody, and a dual-affinity re-targeting antibody (DART).

In some embodiments, the molecule further comprises a second antigen-binding domain that can bind to an effector cell receptor selected from the group consisting of CD3, CD28, CD4, CD8, CD16a, NKG2D, PD-1, CTLA-4, 4-1BB, OX40, ICOS, and CD27. In some embodiments, the second antigen-binding domain can bind to CD3. In some embodiments, the second antigen-binding domain that can bind to CD3 comprises a variable light chain and a variable heavy chain selected from those shown in Table 3. In some embodiments, the second antigen-binding domain that can bind to CD3 comprises or consists of any one of those shown in Table 2 (SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51).

In some embodiments, the molecule is a single-chain diabody (scDb). In some embodiments, the single-chain diabody comprises, in order from N- to C-terminus: (i) an scFv light chain comprising: (a) an scFv light chain CDR1 comprising or consisting of SEQ ID NO: 8, (b) an scFv light chain CDR2 comprising or consisting of SEQ ID NO: 9 or SEQ ID NO: 23, (c) an scFv light chain CDR3 comprising or consisting of SEQ ID NO: 10; (ii) an antigen binding domain that can bind to an effector cell receptor selected from the group consisting of CD3, CD28, CD4, CD8, CD16a, NKG2D, PD-1, CTLA-4, 4-1BB, OX40, ICOS, and CD27; and (iii) an scFv heavy chain comprising: (a) an scFV heavy chain CDR1 comprising or consisting of SEQ ID NO: 17; (b) an scFV heavy chain CDR2 comprising or consisting of SEQ ID NO: 18 or SEQ ID NO: 27; and (c) an scFV heavy chain CDR3 comprising or consisting of SEQ ID NO: 19.

In some embodiments, the single-chain diabody comprises: (a) the scFv light chain CDR2 comprising or consisting of SEQ ID NO: 23; or (b) the scFv heavy chain CDR2 comprising or consisting of SEQ ID NO: 27; or (c) the scFv light chain CDR2 comprising or consisting of SEQ ID NO: 23 and the scFv heavy chain CDR2 comprising or consisting of SEQ ID NO: 27. In some embodiments, the single-chain diabody comprises, in order from N- to C-terminus: (i) an scFv light chain comprising or consisting of SEQ ID NO: 7 or SEQ ID NO: 22; (ii) an antigen binding domain that can bind to an effector cell receptor selected from the group consisting of CD3, CD28, CD4, CD8, CD16a, NKG2D, PD-1, CTLA-4, 4-1BB, OX40, ICOS, and CD27; and (iii) an scFv heavy chain comprising or consisting of SEQ ID NO: 16 or SEQ ID NO: 26. In some embodiments, the single-chain diabody comprises: (a) the scFv light chain comprising or consisting of SEQ ID NO: 7 and the scFv heavy chain comprising or consisting of SEQ ID NO: 26; or (b) the scFv light chain comprising or consisting of SEQ ID NO: 22 and the scFv heavy chain comprising or consisting of SEQ ID NO: 16; or (c) the scFv light chain comprising or consisting of SEQ ID NO: 22 and the scFv heavy chain comprising or consisting of SEQ ID NO: 26.

In some embodiments, the antigen binding domain is a CD3 antigen binding domain. In some embodiments, the CD3 antigen binding domain comprises a variable light chain and a variable heavy chain selected from those shown in Table 3.

In some embodiments, the variable light chain and variable heavy chain of the antigen binding domain are separated by a linker, preferably a 3×G4S linker (SEQ ID NO: 13). In some embodiments, the antigen-binding domain that can bind to CD3 comprises or consists of any one of those shown in Table 2 (SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51).

In some embodiments, the molecule further comprises a first linker between the scFv light chain and the antigen binding domain and a second linker between the antigen binding domain and the scFv heavy chain. In some embodiments, the first linker comprises or consists of G4S (SEQ ID NO: 11) and the second linker comprises or consists of G4S (SEQ ID NO: 15).

Also provided herein are methods for treating a mammal having a cancer expressing a mutant peptide comprising or consisting of HMTEVVRHC (SEQ ID NO: 1), the method comprising: administering to the mammal a molecule of any one of claims 1 to 24. In some embodiments, said mammal is a human. In some embodiments, said cancer is Hodgkin's lymphoma, non-Hodgkin's lymphoma, acute myeloid leukemia, acute lymphoblastic leukemia, multiple myeloma, a myelodysplastic syndrome (MDS), a myeloproliferative disease, lung cancer, pancreatic cancer, gastric cancer, colorectal cancer, ovarian cancer, endometrial cancer, biliary tract cancer, liver cancer, breast cancer, prostate cancer, esophageal cancer, stomach cancer, kidney cancer, bone cancer, soft tissue cancer, head and neck cancer, glioblastoma multiforme, astrocytoma, thyroid cancer, germ cell tumor, or melanoma.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, the term “about” a number refers to that number plus or minus 10% of that number. The term “about” a range refers to that range minus 10% of its lowest value and plus 10% of its greatest value.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows a variant library design. The original H2 scFv was modified at 61 sites across all 6 CDRs with each of the 19 amino acids for a total of 1159 variants. The scFv library was directly synthesized and cloned into the pADL-10b vector for panning by phage display.

FIG. 2 shows sites of amino acid modification in picked panning colonies. SS320 competent cells were infected with phage pools from the end of round 4 and round 5 of panning and were plated for picking single colonies. 100 colonies per round underwent Sanger sequencing and individual variants were identified. High diversity remained at the end of panning with few repeated variants and all CDRs represented in sequenced colonies.

FIG. 3 shows results of screening variants with ammonium thiocyanate and urea. Predicted structurally-relevant phage clones were applied to ELISA plates coated with R175H/A2 pMHC or R175WT pMHC. After initial washing, plates were treated with varying concentrations of NH₄SCN or urea to elute low affinity binders. Variants were assessed by linear regression analysis of ELISA absorbance (A₄₅₀) vs. wash concentration. Slopes for variants with R175H:R175WT binding greater than or equal to the original H2 variant are displayed.

FIG. 4 shows results of thiocyanate-based screening for pooled phage. The phage pool from round 4 of panning underwent screening with 0.5M NH₄SCN washes or control wash (HBSPE). Phage was applied to ELISA plates coated with R175H/A2 monomer and washed with TBST followed by the test wash buffers (SCN or HBSPE). Bound phage was eluted and used to infect SS320 cells to generate more phage. The same protocol was repeated on this single-enriched phage pool. Variant enrichment was tracked using next-generation sequencing. Variants with read fraction in the top 50 percentile and at least two-fold enrichment from the starting round 4 phage pool are displayed. Selected enriched variants were then screened as scDbs similarly to the structurally-predicted variants.

FIG. 5 demonstrates that variant scDbs have increased relative binding to the R175H/HLA-A2 monomer as compared to the original H2 scDb. To confirm that the expressed scDbs bind the R175H/A2 monomer specifically and can bind CD3, scDbs were applied to plates coated with R175H/A2 monomer, R175WT/A2 monomer or the CD3delta/epsilon heterodimer. When normalized to CD3 binding signal, most variant scDbs demonstrated increased relative binding to the R175H/HLA-A2 monomer compared to the original H2-scDb by ELISA. All variants had no R175WT/HLA-A2 binding.

FIG. 6 shows the results of testing scDb sensitivity in peptide-pulsing co-culture. TAP-deficient T2A3 cells were pulsed with varying concentrations of R175H 9mer or R175WT 9mer. scDbs were tested for their ability to activate T cells against peptide-pulsed cells at low pulsing concentrations. T cells were co-cultured at a 2:1 effector:target cell ratio with 1 nM scDb in all conditions. T cells produced high levels of IFNg against R175H-pulsed cells but not R175WT cells for all scDbs. Y57I and F53S scDbs generated an increased IFNg response at 1 nM of peptide compared to the double mutant and original H2 scDb.

FIGS. 7A-7B demonstrate that the H2 variants have a lower EC₅₀ than the original H2 scDb in co-culture with KMS26 cells. 5×10⁴ primary human T cells and 2.5×10⁴ KMS26 target cells with an endogenous p53^{R175H} (TP53 R175H, solid symbols) or p53 knockout (TP53 KO, open symbols) were co-cultured in the presence of 3-fold dilutions of scDb from 10 nM to 0.169 pM overnight. H2 variants generate increased cytotoxicity (FIG. 7A) and interferon gamma response (FIG. 7B) compared to the original H2 scDb at the same concentration of scDb. EC₅₀ values against the R175H cell line were determined by 5-parameter logistic curve fitting (R175H solid lines, p53 KO dotted lines).

FIGS. 8A-8D demonstrate that H2 variants generate strong cytotoxicity against target cell lines with endogenous R175H mutations. Variant scDbs were tested at 0.01 nM, 0.1 nM and 1 nM in co-cultures of human T cells with endogenous R175H HLA-A2+ cell lines at a 2:1 effector to target cell ratio. For all target cell lines (TYKnu, FIG. 8A; KMS26, FIG. 8B; KLE, FIG. 8C; Nalm6, FIG. 8D), the variant scDbs had increased activity over the original H2 scDb. Cytotoxicity at each scDb concentration was compared by ordinary two-way ANOVA with Tukey's multiple comparisons test. Nalm6 R175H is an HLA-A2 positive cell line that was engineered to have the TP53 R175H mutation by CRISPR editing. FIGS. 8A-8C: Bars, from left to right, for each scDb: R175H, 0.01 nM; R175H, 0.1 nM; R175H, 1 nM; p53 KO, 0.01 nM; p53 KO, 0.1 nM; p53 KO, 1 nM; FIG. 8D: Bars, from left to right, for each scDb: R175H, 0.01 nM; R175H, 0.1 nM; R175H, 1 nM; p53 KO, 0.01 nM; p53 KO, 0.1 nM; p53 KO, 1 nM.

FIGS. 9A-9D demonstrate that F53S, Y57I and F53S/Y57I have increased affinity for R175H/HLA-A2 by SPR. Binding affinities for the original H2 (FIG. 9A), F53S (FIG. 9B), Y57I (FIG. 9C) and F53S/Y57I (FIG. 9D) scDbs were measured by surface plasmon resonance (SPR). Chips coated with the R175H/A2 (black dashed line) or R175WT/A2 (light gray dashed line) monomer were exposed to increasing concentrations of scDb. The measured affinities (K_{D}) for the original H2 (FIG. 9A), F53S (FIG. 9B), Y57I (FIG. 9C) and F53S/Y57I (FIG. 9D) are 29.5 nM, 12.9 nM, 6.8 nM and 3.3 nM, respectively.

FIGS. 10A-10I demonstrate that H2 variants can control tumor growth in vivo. FIG. 10A: to test whether the variant scDbs have improved in vivo efficacy compared to the original H2 scDb, 13-15 week old female NSG mice were inoculated with 1×10⁶ luciferase-positive KMS26 cells and 1×10⁷ human T cells two days prior to treatment with continuous infusion pumps containing scDb (scDb dose 0.15 mg/kg/day for 14 days). Treatment groups were randomized on Day −1. Tumor burden was followed every 3 days by bioluminescent imaging using the IVIS system. N=6 mice per group. FIGS. 10B-10F: Total flux measurements were normalized to an injection control fluorescent dye measured over the thorax for each mouse: FIG. 10B, original H2; FIG. 10C, F53S; FIG. 10D, Y57I; FIG. 10E, F53S/Y57I; FIG. 10F: L2 isotype control. FIG. 10G: Bioluminescence images at each time point are displayed. Luminescence was detected over a 60 s exposure time for all time points. FIG. 10H demonstrates that Y57I and F53S/Y57I control KMS26 tumor growth in vivo. A two-way repeated measures ANOVA with the Geissner-Greenhouse correction and Dunnett's multiple comparisons test comparing each treatment scDb to the original H2 scDb at each time point indicates that Y57I and F53S/Y57I control tumor growth better than H2 at treatment days 5 and 8 (P<0.05). Total flux measurements were normalized to an injection control fluorescent dye measured over the thorax for each mouse. FIG. 10I demonstrates that Y57I controls KMS26 tumor growth in vivo. Multiple Mann-Whitney tests of the bioluminescent data from FIGS. 9A-9G indicate that Y57I has improved tumor control at Day 8 compared to the original H2 scDb (P 0.019). In the two weeks following treatment, no Y57I-treated mice had tumor recurrence. N=6 mice per group. Total flux measurements were normalized to an injection control fluorescent dye measured over the thorax for each mouse.

FIGS. 11A-11G indicate that the H2 variants control Nalm6^{R175H} tumor growth in vivo. FIG. 11A: To test whether the H2 variants can better control the faster-growing Nalm6^{R175H} cell line at a lower scDb dose, 7-8 week old female NSG mice were inoculated with 5×10⁵ luciferase-positive Nalm6^{R175H} cells and 1×10⁷ human T cells two days prior to treatment with continuous infusion pumps containing scDb (scDb dose 0.075 mg/kg/day for 14 days). Treatment groups were randomized on Day −1. Tumor burden was followed by bioluminescent imaging using the IVIS system. Two independent experiments were conducted, N=6 per scDb. Results are reported in aggregate, N=12 total. FIGS. 11B-11F: Average radiance values: FIG. 11B, original H2; FIG. 11C, F53S; FIG. 11D, Y57I; FIG. 11E, F53S/Y57I; FIG. 11F: Isotype control. FIG. 11G: Two-way repeated measures ANOVA with Geissner-Greenhouse correction and Dunnett's multiple comparisons test indicate that at treatment day 7, all H2 variants had improved tumor control (H2 vs. F53S P 0.0093, Y57I P=0.0126, F53S/Y57I P=0.0030) and that H2 was superior to the isotype control scDb (P<0.0001). F53S/Y57I had superior tumor control compared to H2 at day 14 by Dunnett's multiple comparisons test (2-way RM ANOVA, P 0.0295).

FIGS. 12A-12E indicate that Y57I scDb controls KMS26 in a delayed treatment model in vivo. FIG. 12A: To compare the anti-tumor efficacy of scDb Y57I in an established tumor model, 7-9 week-old female NSG mice were inoculated with 3.5×10⁵ KMS26 cells and randomized 6 days later on treatment day −1. 7 days after tumor inoculation, mice were treated with 1×10⁷ human T cells intravenously and 0.075 mg/kg/d scDb using a 14-day continuous release pump surgically placed in the mouse peritoneal space. N=5 per group. FIG. 12B-12D: Average radiance values: FIG. 12B: original H2; FIG. 12C: Y57I; FIG. 12D: Isotype. FIG. 12E: Group average radiance. At end of treatment on day 14, a Mann-Whitney test comparing H2 scDb and Y57I scDb indicated that mice treated with Y57I scDb had a lower tumor burden (P=0.0079).

### DETAILED DESCRIPTION

This document relates to methods and materials for assessing a mammal having or suspected of having cancer and/or for treating a mammal having cancer. For example, this document provides methods and materials for using a molecule including one or more antigen-binding domains (e.g., a single-chain variable fragment (scFv)) that can bind to a modified peptide (e.g., a tumor antigen) to treat a mammal having a cancer.

One example of a bispecific antibody targeting an intracellular driver gene is the H2 bispecific antibody, which targets the TP53 R175H mutation presented as a 9mer peptide (mutant peptide HMTEVVRHC (SEQ ID NO: 1); corresponding to positions 168-176 of SEQ ID NO: 3, with a R→H mutation at position 175); WT peptide HMTEVVRRC (SEQ ID NO: 2; corresponding to positions 168-176 of SEQ ID NO: 3)) on HLA-A2. See Hsiue et al., “Targeting a Neoantigen Derived from a Common TP53 Mutation,” Science 371(6533):eabc8697 (2021); see also WO2021/12784, each of which is hereby incorporated by reference in its entirety. The H2 bispecific includes an anti-CD3 binding domain at one end of the molecule and a pMHC-binding domain at the opposite end. Upon binding to the target cell pMHC and CD3 in the T cell receptor complex of a T cell, the bispecific antibody activates the T cell to induce both cytokine release and T cell-mediated killing of the target cell. While this H2 bispecific antibody is able to drive anti-cancer cell killing in vitro and limit tumor growth in vivo, further improvements could allow for improved tumor clearance and performance in vivo. Indeed, others have shown that increasing the binding affinity of bispecific antibodies can significantly improve their potency.

Described herein, among other things, are affinity matured H2 variants that maintain specificity for the R175H mutant peptide having improved performance both in vitro and in vivo identified by a screening method to select variants containing a single amino acid change in the pMHC binding domain and then differentiate specific variants by relative affinity. Variants with high relative affinity identified by this method outperform the original H2 variant both in vitro and in vivo. These variant bispecific antibodies have a higher affinity as measured by surface plasmon resonance (SPR) while maintaining the same specificity for the R175H mutant peptide. These affinity matured H2 variants can also be adapted for use as a chimeric antigen receptor (CAR) or other scFv-based therapy.

Also described herein are methods and compositions for assessing a mammal having cancer or suspected of having cancer and/or treating a mammal having cancer. For example, one or more molecules including one or more antigen-binding domains (e.g., scFvs) that can target (e.g., bind to) the TP53 R175H mutant peptide can be used to assess a mammal having cancer or suspected of having cancer and/or to treat a mammal having a cancer (e.g., a cancer expressing one or more modified peptides). In some cases, the one or more molecules including one or more antigen-binding domains (e.g., scFvs) that can target (e.g., bind to) the TP53 R175H mutant peptide can be used to detect the presence or absence of one or more modified peptides in a sample obtained from a mammal having cancer or suspected of having cancer. In some cases, the one or more molecules including one or more antigen-binding domains (e.g., scFvs) that can target (e.g., bind to) the TP53 R175H mutant peptide can be administered to a mammal having a cancer (e.g., a cancer expressing the modified peptide) to treat the mammal.

As used herein, a modified peptide is a peptide derived from a modified polypeptide. A modified polypeptide can be any appropriate modified polypeptide (e.g., a polypeptide having a disease-causing mutation such as a mutation in an oncogenic or a mutation in a tumor suppressor gene). A modified peptide can have one or more amino acid modifications (e.g., substitutions) relative to a WT peptide (e.g., a peptide derived from a WT polypeptide from which the modified polypeptide is derived). A modified peptide also can be referred to as a mutant peptide. In some cases, a modified peptide can be a tumor antigen. Examples of tumor antigens include, without limitation, MANAs, tumor-associated antigens, and tumor-specific antigens.

A modified peptide can be any appropriate length. In some cases, a modified peptide can be from about 7 amino acids to about 25 amino acids (e.g., from about 8 amino acids to about 25 amino acids, from about 9 amino acids to about 25 amino acids, from about 10 amino acids to about 25 amino acids, from about 11 amino acids to about 25 amino acids, from about 12 amino acids to about 25 amino acids, from about 13 amino acids to about 25 amino acids, from about 15 amino acids to about 25 amino acids, from about 18 amino acids to about 25 amino acids, from about 20 amino acids to about 25 amino acids, from about 7 amino acids to about 22 amino acids, from about 7 amino acids to about 20 amino acids, from about 7 amino acids to about 18 amino acids, from about 7 amino acids to about 15 amino acids, from about 7 amino acids to about 12 amino acids, from about 7 amino acids to about 10 amino acids, from about 7 amino acids to about 9 amino acids, from about 8 amino acids to about 22 amino acids, from about 10 amino acids to about 18 amino acids, from about 12 amino acids to about 15 amino acids, from about 8 amino acids to about 12 amino acids, from about 12 amino acids to about 18 amino acids, from about 18 amino acids to about 22 amino acids, or from about 9 amino acids to about 10 amino acids) in length. For example, a modified peptide can be about 9 amino acids in length. For example, a modified peptide can be about 10 amino acids in length. A modified peptide can be derived from any modified polypeptide. Examples of modified polypeptides from which modified peptides described herein can be derived include, without limitation, p53.

A modified TP53 R175H mutant peptide described herein (e.g., comprising or consisting of SEQ ID NO: 1) can be in a complex with any appropriate HLA. An HLA can be any appropriate HLA allele. In some cases, an HLA can be a class I HLA (e.g., HLA-A, HLA-B, and HLA-C) allele. In some cases, an HLA can be a class II HLA (e.g., HLA-DP, HLA-DM, HLA-DOA, HLA-DOB, HLA-DQ, and HLA-DR) allele. Examples of HLA alleles that a modified peptide described herein can complex with include, without limitation, HLA-A1 and HLA-A2. Exemplary HLA alleles for particular modified peptides are shown in Table 1. For example a modified TP53 R175H mutant peptide described herein (e.g., comprising or consisting of SEQ ID NO: 1) can be in a complex with HLA-A2 and 02M.

Described herein are molecules including one or more antigen-binding domains (e.g., scFvs) that can bind to a modified TP53 R175H mutant peptide described herein (e.g., comprising or consisting of SEQ ID NO: 1). In some cases, a molecule including one or more antigen-binding domains that can bind to a modified TP53 R175H mutant peptide described herein (e.g., comprising or consisting of SEQ ID NO: 1) does not target (e.g., does not bind to) an uncomplexed modified peptide described herein (e.g., comprising or consisting of e.g., a modified peptide described herein that is not present in a complex (e.g., a peptide-HLA-02M complex)). In some cases, a molecule including one or more antigen-binding domains that can bind to a modified TP53 R175H mutant peptide described herein (e.g., comprising or consisting of SEQ ID NO: 1) does not target (e.g., does not bind to) a WT peptide (e.g., a peptide derived from a WT polypeptide from which a modified polypeptide is derived).

A molecule including one or more antigen-binding domains (e.g., scFvs) that can bind to a modified TP53 R175H mutant peptide described herein (e.g., comprising or consisting of SEQ ID NO: 1) can be any appropriate type of molecule. In some cases, a molecule can be a monovalent molecule (e.g., containing a single antigen-binding domain). In some cases, a molecule can be a multivalent molecule (e.g., containing two or more antigen-binding domains and simultaneously targeting two or more antigens). For example, a bispecific molecule can include two antigen-binding domains, a trispecific molecule can include three antigen-binding domains, a quadruspecific molecule can include four antigen-binding domains, etc. Examples of molecules that contain antigen-binding domains include, without limitation, antibodies, antibody fragments, scFvs, chimeric antigen receptors (CARs), T cell receptors (TCRs), TCR mimics, tandem scFvs, bispecific T cell engagers, diabodies, scDbs, scFv-Fcs, bispecific antibodies, bispecific single-chain Fcs, dual-affinity re-targeting antibodies (DARTs), and any other molecule that includes at least one variable heavy chain (VH) and at least one variable light chain (VL). Any of these molecules can be used in accordance with materials and methods described herein. In some cases, an antigen-binding domain can be a scFv. For example, a molecule including one or more antigen-binding domains (e.g., one or more scFvs) that can bind to a modified peptide described herein can be a CAR. For example, a molecule including two scFvs that can bind to a modified peptide described herein can be a single-chain diabody (scDb).

In some cases, when a molecule including one or more antigen-binding domains (e.g., scFvs) that can bind to a modified TP53 R175H mutant peptide described herein (e.g., comprising or consisting of SEQ ID NO: 1) is a multivalent molecule (e.g., a bispecific molecule), a first antigen-binding domain can bind to a modified peptide described herein and a second antigen-binding domain can bind to an effector cell (e.g., an antigen present on an effector cell). Examples of effector cells include, without limitation, T cells, natural killer (NK) cells, natural killer T (NKT) cells, B cells, plasma cells, macrophages, monocytes, microglia, dendritic cells, neutrophils, fibroblasts, and mast cells. Examples of antigens present on effector cells include, without limitation, CD3, CD4, CD8, CD28, NKG2D, PD-1, CTLA-4, 4-1BB, OX40, ICOS, CD27, Fc receptors (e.g., CD16a), and any other effector cell surface receptors. In some cases, a molecule described herein can include a first antigen-binding domain that can bind to a modified TP53 R175H mutant peptide described herein (e.g., comprising or consisting of SEQ ID NO: 1) and a second antigen-binding domain that can bind to an antigen present on a T cell (e.g., CD3). In some cases, sequences (e.g., scFv sequences) that can bind to CD3 can be as shown in Table 2 or Table 3. In some cases, sequences (e.g., scFv sequences) that can bind to CD3 can be as described elsewhere (see, e.g., Rodrigues et al., 1992 Int J Cancer Suppl. 7:45-50; Shalaby et al., 1992 J Exp Med. 175:217-25; Brischwein et al., 2006 Mol Immunol. 43:1129-43; Li et al., 2005 Immunology. 116:487-98; WO2012162067; US20070065437; US20070065437; US20070065437; US20070065437; US20070065437; and US20070065437). In some cases, a molecule described herein can include a first antigen-binding domain that can bind to a modified peptide described herein and a second antigen-binding domain that can bind to an antigen present on a NK cell (e.g., CD16a or NKG2D). In some cases, sequences (e.g., scFv sequences) that can bind to CD16a can be as shown in Table 4. By binding both a modified peptide and the effector cell, the multivalent molecule can bring the cell expressing a modified peptide (e.g., as part of the HLA complex) into proximity with the effector cell, permitting the effector cell to act on the cell expressing a modified peptide.

In some cases, when a molecule including one or more antigen-binding domains (e.g., scFvs) that can bind to a modified TP53 R175H mutant peptide described herein (e.g., comprising or consisting of SEQ ID NO: 1) is a multivalent molecule (e.g., a bispecific molecule), a molecule can be in any appropriate format which includes at least one VH and at least one VL. For example, a VH and a VL can be in any appropriate orientation. In some cases, a VH can be N-terminal to the VL. In some cases, a VH can be C-terminal to the VL. In some cases, a linker amino acid sequence can be positioned between the VH and VL.

In some cases, when a bispecific molecule includes tandem scFvs, the tandem scFvs can be in any appropriate orientation. Examples of tandem scFv orientations including scFv-A and scFv-B include, without limitation, VLA-LL-VHA-SL-VLB-LL-VHB, VLA-LL-VHA-SL-VHB-LL-VLB, VHA-LL-VLA-SL-VLB-LL-VHB, VHA-LL-VLA-SL-VHB-LL-VLB, VLB-LL-VHB-SL-VLA-LL-VHA, VLB-LL-VHB-SL-VHA-LL-VLA, VHB-LL-VLB-SL-VLA-LL-VHA, and VHB-LL-VLB-SL-VHA-LL-VLA, where SL is a short linker and LL is a long linker. A short linker can be from about 3 amino acids to about 10 amino acids in length. A short linker can include any appropriate amino acids (e.g., glycines and serines) in any appropriate combination. A long linker can be from about 10 amino acids to about 25 amino acids in length. A long linker can include any appropriate amino acids (e.g., glycines and serines) in any appropriate combination.

In some cases, when a bispecific molecule is a diabody, the diabody can be in any appropriate orientation. Examples of diabody orientations including scFv-A and scFv-B include, without limitation, VLA-SL-VHB and VLB-SL-VHA, VLA-SL-VLB and VHB-SL-VHA, VHA-SL-VLB and VHB-SL-VLA, VLB-SL-VHA and VLA-SL-VHB, VLB-SL-VLA and VHA-SL-VHB, and VHB-SL-VLA and VHA-SL-VLB, where SL is a short linker. A short linker can be from about 3 amino acids to about 10 amino acids in length. A short linker can include any appropriate amino acids (e.g., glycines and serines) in any appropriate combination.

In some cases, when a bispecific molecule is a scDb, the scDb can be in any appropriate orientation. Examples of scDb orientations including scFv-A and scFv-B include, without limitation, VLA-SL-VHB-LL-VLB-SL-VHA, VHA-SL-VLB-LL-VHB-SL-VLA, VLA-SL-VLB-LL-VHB-SL-VHA, VHA-SL-VHB-LL-VLB-SL-VLA, VLB-SL-VHA-LL-VLA-SL-VHB, VHB-SL-VLA-LL-VHA-SL-VLB, VLB-SL-VLA-LL-VHA-SL-VHB, and VHB-SL-VHA-LL-VLA-SL-VLB, where SL is a short linker and LL is a long linker. A short linker can be from about 3 amino acids to about 10 amino acids in length. A short linker can include any appropriate amino acids (e.g., glycines and serines) in any appropriate combination. A long linker can be from about 10 amino acids to about 25 amino acids in length. A long linker can include any appropriate amino acids (e.g., glycines and serines) in any appropriate combination.

In some cases, when a bispecific molecule is a scFv-Fc, the scFv-Fc can be in any appropriate orientation. Examples of scFv-Fc orientations including scFv-Fc-A, scFv-Fc-B, and an Fc domain include, without limitation, VLA-LL-VHA-hinge-Fc and VLB-LL-VHB-hinge-Fc, VHA-LL-VLA-hinge-Fc and VHB-LL-VLB-hinge-Fc, VLA-LL-VHA-hinge-Fc and VHB-LL-VLB-hinge-Fc, VHA-LL-VLA-hinge-Fc and VLB-LL-VHB-hinge-Fc, where LL is a long linker. A long linker can be from about 10 amino acids to about 25 amino acids in length. A long linker can include any appropriate amino acids (e.g., glycines and serines) in any appropriate combination. In some cases, an Fc domain in a scFv-Fc can include one or more modifications to increase heterodimerization and/or to decrease homodimerization of the scFv-Fc. In some cases, an Fc domain in a scFv-Fc can exclude a hinge domain. In some cases, an Fc domain in a scFv-Fc can be at the N-terminus of the scFv.

In some cases, when a bispecific molecule is a bispecific single-chain Fc, the bispecific single-chain Fc can be in any appropriate orientation. Examples of bispecific single-chain Fc orientations include, without limitation, VLA-LL-VHA-SL-VHB-LL-VLB-SL-hinge-CH2-CH3-LL-hinge-CH2-CH3, VLA-LL-VHA-SL-VLB-LL-VHB-SL-hinge-CH2-CH3-LL-hinge-CH2-CH3, VHA-LL-VLA-SL-VLB-LL-VHB-SL-hinge-CH2-CH3-LL-hinge-CH2-CH3, VHA-LL-VLA-SL-VHB-LL-VLB-SL-hinge-CH2-CH3-LL-hinge-CH2-CH3, and VLA-SL-VHB-LL-VLB-VHA-SL-hinge-CH2-CH3-LL-hinge-CH2-CH3, where SL is a short linker and LL is a long linker. A short linker can be from about 3 amino acids to about 8 amino acids in length. A short linker can include any appropriate amino acids (e.g., glycines and serines) in any appropriate combination. A long linker can be from about 10 amino acids to about 25 amino acids in length. A long linker can include any appropriate amino acids (e.g., glycines and serines) in any appropriate combination. Any appropriate Fc domain can be used in a bispecific single-chain Fc. In some cases, an Fc domain can include an amino acid sequence derived from an IgG (e.g., a natural IgG). In some cases, an Fc domain can include an amino acid sequence that includes one or more modifications (e.g., one or more modifications to increase stability of the molecule and/or to increase or decrease binding to one or more Fc receptors). In some cases, an Fc domain that can be used in a bispecific single-chain Fc can exclude a hinge domain. In some cases, an Fc domain that can be used in a bispecific single-chain Fc can be at the N-terminus of the scFvs. In some cases, an Fc domain that can be used in a bispecific single-chain Fc can be as described elsewhere (see, e.g., International Patent Application Publication No. WO 2017/134134 A1 at, for example, SEQ ID NOs: 25-32; and International Patent Application Publication No. WO 2017/134158 A1 at, for example, Table 38; and SEQ ID NOs: 25-32).

A molecule including one or more antigen-binding domains (e.g., scFvs) that can bind to a modified TP53 R175H mutant peptide described herein (e.g., comprising or consisting of SEQ ID NO: 1) can include any appropriate complementarity determining regions (CDRs). For example, a molecule including one or more antigen-binding domains that can bind to a modified peptide described herein can include a variable heavy chain (VH) having three VH complementarity determining regions (CDR-VHs) and a variable light chain (VL) having three VL CDRs (CDR-VLs).

In some cases, the molecule that can bind to a modified TP53 R175H mutant peptide described herein (e.g., comprising or consisting of SEQ ID NO: 1) comprises one or more mutations relative to H2 (SEQ ID NO: 4) selected from the group consisting of Y57I, S92N, A31S, S95T, S92E, S95D, S95K, R24D, V29D, F53S, S26P, V29E, Q90D, and Y57L. In some cases, the molecule that can bind to a modified TP53 R175H mutant peptide described herein (e.g., comprising or consisting of SEQ ID NO: 1) includes one, two, three, four, five, six, seven, eight, nine, ten, eleven, twelve, thirteen, fourteen, or fourteen mutations (relative to H2, SEQ ID NO: 4) selected from the group consisting of Y57I, S92N, A31S, S95T, S92E, S95D, S95K, R24D, V29D, F53S, S26P, V29E, Q90D, and Y57L. In some cases, the molecule that can bind to a modified TP53 R175H mutant peptide described herein (e.g., comprising or consisting of SEQ ID NO: 1) comprises F53S and/or Y57I mutations relative to H2 (SEQ ID NO: 4). Thus, a molecule that can bind to a modified TP53 R175H mutant peptide described herein (e.g., comprising or consisting of SEQ ID NO: 1) can include one of each of the H2 CDRs (CDR-VL1: SEQ ID NO: 8; CDR-VL2: SEQ ID NO: 9; CDR-VL3: SEQ ID NO: 10; CDR-VH1: SEQ ID NO: 17; CDR-VH2: SEQ ID NO: 18; and CDR-VH3: SEQ ID NO: 19), so long as the molecule comprises one, two, three, four, five, six, seven, eight, nine, ten, eleven, twelve, thirteen, fourteen, or fourteen mutations (relative to H2, SEQ ID NO: 4) selected from the group consisting of Y57I, S92N, A31S, S95T, S92E, S95D, S95K, R24D, V29D, F53S, S26P, V29E, Q90D, and Y57L. In some cases, the mutation(s) are F53S and/or Y57I.

For example, a molecule that can bind to a modified TP53 R175H mutant peptide described herein (e.g., comprising or consisting of SEQ ID NO: 1) can include one of each of the CDRs set forth below:
CDR-VL1: SEQ ID NO: 8;
CDR-VL2: SEQ ID NO: 9 or SEQ ID NO: 23;
CDR-VL3: SEQ ID NO: 10;
CDR-VH1: SEQ ID NO: 17;
CDR-VH2: SEQ ID NO: 18 or SEQ ID NO: 27; and
CDR-VH3: SEQ ID NO: 19,
   wherein:
i) CDR-VL2 is SEQ ID NO: 23; or
ii) CDR-VH2 is SEQ ID NO: 27; or
iii) CDR-VL2 is SEQ ID NO: 23 and CDR-VH2 is SEQ ID NO: 27.

For example, a molecule that can bind to a modified TP53 R175H mutant peptide described herein (e.g., comprising or consisting of SEQ ID NO: 1) can include one of each of the CDRs set forth below:
CDR-VL1: SEQ ID NO: 8;
CDR-VL2: SEQ ID NO: 23;
CDR-VL3: SEQ ID NO: 10;
CDR-VH1: SEQ ID NO: 17;
CDR-VH2: SEQ ID NO: 18 or SEQ ID NO: 27; and
CDR-VH3: SEQ ID NO: 19.

For example, a molecule that can bind to a modified TP53 R175H mutant peptide described herein (e.g., comprising or consisting of SEQ ID NO: 1) can include one of each of the CDRs set forth below:
CDR-VL1: SEQ ID NO: 8;
CDR-VL2: SEQ ID NO: 23;
CDR-VL3: SEQ ID NO: 10;
CDR-VH1: SEQ ID NO: 17;
CDR-VH2: SEQ ID NO: 18; and
CDR-VH3: SEQ ID NO: 19.

For example, a molecule that can bind to a modified TP53 R175H mutant peptide described herein (e.g., comprising or consisting of SEQ ID NO: 1) can include one of each of the CDRs set forth below:
CDR-VL1: SEQ ID NO: 8;
CDR-VL2: SEQ ID NO: 23;
CDR-VL3: SEQ ID NO: 10;
CDR-VH1: SEQ ID NO: 17;
CDR-VH2: SEQ ID NO: 27; and
CDR-VH3: SEQ ID NO: 19.

For example, a molecule that can bind to a modified TP53 R175H mutant peptide described herein (e.g., comprising or consisting of SEQ ID NO: 1) can include one of each of the CDRs set forth below:
CDR-VL1: SEQ ID NO: 8;
CDR-VL2: SEQ ID NO: 9 or SEQ ID NO: 23;
CDR-VL3: SEQ ID NO: 10;
CDR-VH1: SEQ ID NO: 17;
CDR-VH2: SEQ ID NO: 27; and
CDR-VH3: SEQ ID NO: 19.

For example, a molecule that can bind to a modified TP53 R175H mutant peptide described herein (e.g., comprising or consisting of SEQ ID NO: 1) can include one of each of the CDRs set forth below:
CDR-VL1: SEQ ID NO: 8;
CDR-VL2: SEQ ID NO: 9;
CDR-VL3: SEQ ID NO: 10;
CDR-VH1: SEQ ID NO: 17;
CDR-VH2: SEQ ID NO: 27; and
CDR-VH3: SEQ ID NO: 19.

For example, a molecule that can bind to a modified TP53 R175H mutant peptide described herein (e.g., comprising or consisting of SEQ ID NO: 1) can include one of each of the CDRs set forth below:
CDR-VL1: SEQ ID NO: 8;
CDR-VL2: SEQ ID NO: 23;
CDR-VL3: SEQ ID NO: 10;
CDR-VH1: SEQ ID NO: 17;
CDR-VH2: SEQ ID NO: 27; and
CDR-VH3: SEQ ID NO: 19.

In some cases, a molecule including one or more antigen-binding domains (e.g., scFvs) that can bind to a modified TP53 R175H mutant peptide described herein (e.g., comprising or consisting of SEQ ID NO: 1) can include any appropriate set of CDR sequences (e.g., any of the CDR sequence sets described herein).

A molecule including one or more antigen-binding domains (e.g., scFvs) that can bind to a modified TP53 R175H mutant peptide described herein (e.g., comprising or consisting of SEQ ID NO: 1) can include any appropriate sequence. For example, a molecule that can bind to a modified TP53 R175H mutant peptide described herein (e.g., comprising or consisting of SEQ ID NO: 1) can include, without limitation, the scFv sequence set forth in any one of SEQ ID NO: 20, SEQ ID NO: 24, or SEQ ID NO: 28. Examples of sequences (e.g., scFv sequences) that can bind to particular modified peptides are shown in Table 1. In some cases, a molecule including one or more antigen-binding domains (e.g., scFvs) that can bind to a modified TP53 R175H mutant peptide described herein (e.g., comprising or consisting of SEQ ID NO: 1) can have a sequence that deviates from a sequence shown in Table 1, sometimes referred to as a variant sequence. For example, a molecule including one or more antigen-binding domains that can bind to a modified peptide described herein can have at least 75% sequence identity (e.g., at least 80% sequence identity, at least 85% sequence identity, at least 90% sequence identity, at least 95% sequence identity, at least 96% sequence identity, at least 97% sequence identity, at least 98% sequence identity, at least 99% sequence identity, or more) to any of the sequences shown in Table 1, provided the variant sequence maintains the ability to bind to a modified peptide described herein. For example, a molecule including one or more antigen-binding domains that can bind to a modified peptide described herein can have one or more (e.g., one, two, three, four, five, six, seven, eight, nine, ten, or more) modifications (e.g., one or more amino acid substitutions) as compared to the sequences shown in Table 1, provided the variant sequence maintains the ability to bind to a modified peptide described herein. In some cases, a molecule including one or more antigen-binding domains that can bind to a modified peptide described herein can include any appropriate set of CDR sequences described herein, and any sequence deviations from a sequence shown in Table 1 can be in the scaffold sequence(s).

A molecule including one or more antigen-binding domains (e.g., scFvs) that can bind to a modified TP53 R175H mutant peptide described herein (e.g., comprising or consisting of SEQ ID NO: 1) can be attached (e.g., covalently or non-covalently attached) to a label (e.g., a detectable label). A detectable label can be any appropriate label. In some cases, a label can be used to assist in detecting the presence or absence of one or more modified peptides described herein. For example, a molecule described herein that is labelled can be used in vitro to detect cancer cells (e.g., cancer cells expressing a modified peptide described herein) in a sample obtained from a mammal. In some cases, a label (e.g., a detectable label) can be used to assist in determining the location of one or more modified peptides described herein. For example, molecule described herein that is labelled can be used in vivo to monitor anti-tumor therapy and/or to detect cancer cells (e.g., cancer cells expressing a modified peptide described herein) in a mammal. Examples of labels that can be attached to a molecule described herein include, without limitation, radionuclides, contrast agents used in magnetic resonance imaging (MRI), computed tomography (CT), ultrasound (US), and other imaging modalities, chromophores, enzymes, and fluorescent molecules (e.g., green fluorescent protein and near-IR fluorescence).

A molecule including one or more antigen-binding domains (e.g., scFvs) that can bind to a modified TP53 R175H mutant peptide described herein (e.g., comprising or consisting of SEQ ID NO: 1)) can be attached (e.g., covalently or non-covalently attached) to a therapeutic agent. A therapeutic agent can be any therapeutic agent. In some cases, a therapeutic agent can be an anti-cancer agent. Examples of therapeutic agents that can be attached to a molecule described herein include, without limitation, anti-cancer agents such as monomethyl auristatin E (MMAE), monomethyl auristatin F (MMAF), maytansine, mertansine/emtansine (DM1), ravtansine/soravtansine (DM4), SN-38, calicheamicin, D6.5, dimeric pyrrolobenzodiazepines (PBDs), α-amantin (AAMT), PNU-159682, ricin, pseudomonas exotoxin A, diphtheria toxin, and gelonin.

This document also provides methods for using one or more molecules including one or more antigen-binding domains (e.g., scFvs) that can bind to a modified TP53 R175H mutant peptide described herein (e.g., comprising or consisting of SEQ ID NO: 1). For example, one or more molecules including one or more antigen-binding domains (e.g., scFvs) that can target (e.g., bind to) a modified TP53 R175H mutant peptide described herein (e.g., comprising or consisting of SEQ ID NO: 1) can be used to assess a mammal having cancer or suspected of having cancer and/or to treat a mammal having a cancer (e.g., a cancer expressing a modified TP53 R175H mutant peptide described herein (e.g., comprising or consisting of SEQ ID NO: 1)). In some cases, one or more molecules includes one or more antigen-binding domains that can bind to a modified TP53 R175H mutant peptide described herein (e.g., comprising or consisting of SEQ ID NO: 1) can be used to detect the presence or absence of a modified TP53 R175H mutant peptide described herein (e.g., comprising or consisting of SEQ ID NO: 1) in a sample obtained from a mammal having cancer or suspected of having cancer. In some cases, one or more molecules including one or more antigen-binding domains that can bind to a modified TP53 R175H mutant peptide described herein (e.g., comprising or consisting of SEQ ID NO: 1) can be administered to a mammal having a cancer (e.g., a cancer expressing the modified peptide) to treat the mammal. Administration of one or more molecules including one or more antigen-binding domains that can bind to a modified TP53 R175H mutant peptide described herein (e.g., comprising or consisting of SEQ ID NO: 1) to a mammal (e.g., human) having a cancer can be effective to treat the mammal.

Any type of mammal can be assessed and/or treated as described herein. Examples of mammals that can be assessed and/or treated as described herein include, without limitation, primates (e.g., humans and non-human primates such as chimpanzees, baboons, or monkeys), dogs, cats, pigs, sheep, rabbits, mice, and rats. In some cases, a mammal can be a human.

A mammal can be assessed and/or treated for any appropriate cancer. In some cases, a cancer can express a modified TP53 R175H mutant peptide described herein (e.g., comprising or consisting of SEQ ID NO: 1). A cancer can be a primary cancer. A cancer can be a metastatic cancer. A cancer can include one or more solid tumors. A cancer can include one or more non-solid tumors. Examples of cancers that can be assessed as described herein (e.g., comprising or consisting of e.g., based at least in part on the presence of one or more modified peptides described herein) and/or treated as described herein (e.g., comprising or consisting of e.g, by administering one or more molecules including one or more antigen-binding domains (e.g., scFvs) that can bind to a modified peptide described herein) include, without limitation, blood cancers (e.g., Hodgkin's lymphoma, non-Hodgkin's lymphoma, acute myeloid leukemia (AML), acute lymphoblastic leukemia (ALL), multiple myeloma, MDS, and myeloproliferative diseases), lung cancers, pancreatic cancers, gastric cancers, colon cancers (e.g., colorectal cancers), ovarian cancers, endometrial cancers, biliary tract cancers, liver cancers, bone and soft tissue cancers (e.g., sarcomas), breast cancers, prostate cancers, esophageal cancers, stomach cancers, kidney cancers, head and neck cancers, brain cancers (e.g., glioblastoma multiforme and astrocytomas), thyroid cancers, germ cell tumors, and melanomas.

When assessing a mammal having cancer or suspected of having cancer, one or more molecules including one or more antigen-binding domains (e.g., scFvs) that can bind to a modified TP53 R175H mutant peptide described herein (e.g., comprising or consisting of SEQ ID NO: 1) can be used to assess for the presence or absence of one or more modified peptides described herein. For example, the presence, absence, or level of a modified TP53 R175H mutant peptide described herein (e.g., comprising or consisting of SEQ ID NO: 1) in a sample obtained from a human can be used to determine whether or not the human has a cancer. In some cases, the presence of a modified TP53 R175H mutant peptide described herein (e.g., comprising or consisting of SEQ ID NO: 1) in a sample obtained from a mammal can be used to identify the mammal as having a cancer. For example, a mammal can be identified as having a cancer when a sample obtained from the mammal has a modified TP53 R175H mutant peptide described herein (e.g., comprising or consisting of SEQ ID NO: 1).

Any appropriate sample obtained from a mammal can be assessed for the presence, absence, or level of a modified TP53 R175H mutant peptide described herein (e.g., comprising or consisting of SEQ ID NO: 1). For example, biological samples such as tissue samples (e.g., breast tissue, and cervical tissue such as from a Papanicolaou (Pap) test), fluid samples (e.g., blood, serum, plasma, urine, saliva, sputum, and cerebrospinal fluid), and solid samples (e.g. stool) can be obtained from a mammal and assessed for the presence, absence, or level of a modified TP53 R175H mutant peptide described herein (e.g., comprising or consisting of SEQ ID NO: 1). Any appropriate method can be used to detect the presence, absence, or level of a modified TP53 R175H mutant peptide described herein (e.g., comprising or consisting of SEQ ID NO: 1). For example, sequencing techniques including, but not limited to, Sanger sequencing, chemical sequencing, nanopore sequencing, sequencing by ligation (SOLiD sequencing), sequencing with mass spectrometry, whole exome sequencing, whole genome sequencing, and/or next-generation sequencing can be used to determine the presence, absence, or level of a modified TP53 R175H mutant peptide described herein (e.g., comprising or consisting of SEQ ID NO: 1) in a sample obtained from a mammal.

When treating a mammal having cancer, one or more molecules including one or more antigen-binding domains (e.g., scFvs) that can bind to a modified TP53 R175H mutant peptide described herein (e.g., comprising or consisting of SEQ ID NO: 1) can be administered to a mammal having cancer to treat the mammal. In some cases, a mammal can have a cancer expressing a modified TP53 R175H mutant peptide described herein (e.g., comprising or consisting of SEQ ID NO: 1). For example, one or more molecules including one or more antigen-binding domains that can bind to a modified TP53 R175H mutant peptide described herein (e.g., comprising or consisting of SEQ ID NO: 1) can be administered to a mammal having a cancer expressing that modified peptide to treat the mammal. For example, one or more molecules including one or more scFvs that can bind to a modified TP53 R175H mutant peptide described herein (e.g., comprising or consisting of SEQ ID NO: 1) (e.g., one or more scDbs) can be administered to a mammal having a cancer expressing that modified peptide to treat the mammal.

In some cases, one or more molecules including one or more antigen-binding domains (e.g., scFvs) that can bind to a modified TP53 R175H mutant peptide described herein (e.g., comprising or consisting of SEQ ID NO: 1) can be administered to a mammal (e.g., a mammal having a cancer) once or multiple times over a period of time ranging from days to weeks.

In some cases, one or more molecules including one or more antigen-binding domains (e.g., scFvs) that can bind to a modified TP53 R175H mutant peptide described herein (e.g., comprising or consisting of SEQ ID NO: 1) can be formulated into a composition (e.g., a pharmaceutically acceptable composition) for administration to a mammal (e.g., a mammal having a cancer). For example, one or more antigen-binding domains that can bind to a modified TP53 R175H mutant peptide described herein (e.g., comprising or consisting of SEQ ID NO: 1) can be formulated together with one or more pharmaceutically acceptable carriers (additives), excipients, and/or diluents. In some cases, a pharmaceutically acceptable carrier, excipient, or diluent can be a naturally occurring pharmaceutically acceptable carrier, excipient, or diluent. In some cases, a pharmaceutically acceptable carrier, excipient, or diluent can be a non-naturally occurring (e.g., an artificial or synthetic) pharmaceutically acceptable carrier, excipient, or diluent. Examples of pharmaceutically acceptable carriers, excipients, and diluents that can be used in a composition described herein include, without limitation, sucrose, lactose, starch (e.g., starch glycolate), cellulose, cellulose derivatives (e.g., modified celluloses such as microcrystalline cellulose and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, benzyl alcohol, lysine hydrochloride, trehalose dihydrate, sodium hydroxide, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, lecithin, and corn oil. In some cases, a pharmaceutically acceptable carrier, excipient, or diluent can be an antiadherent, a binder, a colorant, a disintegrant, a flavor (e.g., a natural flavor such as a fruit extract or an artificial flavor), a glidant, a lubricant, a preservative, a sorbent, and/or a sweetener.

A composition (e.g., a pharmaceutical composition) containing one or more molecules including one or more antigen-binding domains (e.g., scFvs) that can bind to a modified TP53 R175H mutant peptide described herein (e.g., comprising or consisting of SEQ ID NO: 1) can be formulated into any appropriate dosage form. Examples of dosage forms include solid or liquid forms including, without limitation, gums, capsules, tablets (e.g., chewable tablets, and enteric coated tablets), suppositories, liquids, enemas, suspensions, solutions (e.g., sterile solutions), sustained-release formulations, delayed-release formulations, pills, powders, and granules.

A composition containing one or more molecules including one or more antigen-binding domains (e.g., scFvs) that can bind to a modified TP53 R175H mutant peptide described herein (e.g., comprising or consisting of SEQ ID NO: 1) can be designed for oral, parenteral (including subcutaneous, intramuscular, intravenous, and intradermal), or intratumoral administration. Compositions suitable for parenteral administration include aqueous and non-aqueous sterile injection solutions that can contain anti-oxidants, buffers, bacteriostats, and solutes that render the formulation isotonic with the blood of the intended recipient. The formulations can be presented in unit-dose or multi-dose containers, for example, sealed ampules and vials, and may be stored in a freeze dried (lyophilized) condition requiring only the addition of the sterile liquid carrier, for example, water for injections, immediately prior to use. Extemporaneous injection solutions and suspensions may be prepared from sterile powders, granules, and tablets.

A composition containing one or more molecules including one or more antigen-binding domains (e.g., scFvs) that can bind to a modified TP53 R175H mutant peptide described herein (e.g., comprising or consisting of SEQ ID NO: 1) can be administered using any appropriate technique and to any appropriate location. A composition including one or more molecules including one or more antigen-binding domains (e.g., scFvs) that can bind to a modified TP53 R175H mutant peptide described herein (e.g., comprising or consisting of SEQ ID NO: 1) can be administered locally (e.g., intratumorally) or systemically. For example, a composition provided herein can be administered locally by intratumoral administration (e.g., injection into tumors) or by administration into biological spaces infiltrated by tumors (e.g. intraspinal administration, intracerebellar administration, intraperitoneal administration and/or pleural administration). For example, a composition provided herein can be administered systemically by oral administration or by intravenous administration (e.g., injection or infusion) to a mammal (e.g., a human).

Effective doses can vary depending on the risk and/or the severity of the cancer, the route of administration, the age and general health condition of the subject, excipient usage, the possibility of co-usage with other therapeutic treatments such as use of other agents, and the judgment of the treating physician. An effective amount of a composition containing one or more molecules including one or more antigen-binding domains (e.g., scFvs) that can bind to a modified TP53 R175H mutant peptide described herein (e.g., comprising or consisting of SEQ ID NO: 1) can be any amount that treats a cancer present within the subject without producing significant toxicity to the subject. If a particular subject fails to respond to a particular amount, then the amount of one or more molecules including one or more antigen-binding domains (e.g., scFvs) that can bind to a modified TP53 R175H mutant peptide described herein (e.g., comprising or consisting of SEQ ID NO: 1) can be increased (e.g., by two-fold, three-fold, four-fold, or more). After receiving this higher amount, the mammal can be monitored for both responsiveness to the treatment and toxicity symptoms, and adjustments made accordingly. The effective amount can remain constant or can be adjusted as a sliding scale or variable dose depending on the subject's response to treatment. Various factors can influence the actual effective amount used for a particular application. For example, the frequency of administration, duration of treatment, use of multiple treatment agents, route of administration, and severity of the condition (e.g., cancer) may require an increase or decrease in the actual effective amount administered.

The frequency of administration of one or more molecules including one or more antigen-binding domains (e.g., scFvs) that can bind to a modified TP53 R175H mutant peptide described herein (e.g., comprising or consisting of SEQ ID NO: 1) can be any frequency that effectively treats a mammal having a cancer without producing significant toxicity to the mammal. For example, the frequency of administration of one or more molecules including one or more antigen-binding domains (e.g., scFvs) that can bind to a modified TP53 R175H mutant peptide described herein (e.g., comprising or consisting of SEQ ID NO: 1) can be from about two to about three times a week to about two to about three times a year. In some cases, a subject having cancer can receive a single administration of one or more antibodies described herein. The frequency of administration of one or more molecules including one or more antigen-binding domains (e.g., scFvs) that can bind to a modified TP53 R175H mutant peptide described herein (e.g., comprising or consisting of SEQ ID NO: 1) can remain constant or can be variable during the duration of treatment. A course of treatment with a composition containing one or more molecules including one or more antigen-binding domains (e.g., scFvs) that can bind to a modified TP53 R175H mutant peptide described herein (e.g., comprising or consisting of SEQ ID NO: 1) can include rest periods. For example, a composition containing one or more molecules including one or more antigen-binding domains (e.g., scFvs) that can bind to a modified TP53 R175H mutant peptide described herein (e.g., comprising or consisting of SEQ ID NO: 1) can be administered every other month over a two-year period followed by a six-month rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., cancer) may require an increase or decrease in administration frequency.

An effective duration for administering a composition containing one or more molecules including one or more antigen-binding domains (e.g., scFvs) that can bind to a modified TP53 R175H mutant peptide described herein (e.g., comprising or consisting of SEQ ID NO: 1) can be any duration that effectively treats a cancer present within the mammal without producing significant toxicity to the mammal. In some cases, the effective duration can vary from several months to several years. In general, the effective duration for treating a mammal having a cancer can range in duration from about one or two months to five or more years. Multiple factors can influence the actual effective duration used for a particular treatment. For example, an effective duration can vary with the frequency of administration, effective amount, use of multiple treatment agents, route of administration, and severity of the condition being treated.

In certain instances, a cancer within a mammal can be monitored to evaluate the effectiveness of the cancer treatment. Any appropriate method can be used to determine whether or not a mammal having cancer is treated. For example, imaging techniques or laboratory assays can be used to assess the number of cancer cells and/or the size of a tumor present within a mammal. For example, imaging techniques or laboratory assays can be used to assess the location of cancer cells and/or a tumor present within a mammal.

In some cases, one or more molecules including one or more antigen-binding domains (e.g., scFvs) that can bind to a modified TP53 R175H mutant peptide described herein (e.g., comprising or consisting of SEQ ID NO: 1) can be administered to a mammal having a cancer as a combination therapy with one or more additional cancer treatments (e.g., anti-cancer agents). A cancer treatment can include any appropriate cancer treatments. In some cases, a cancer treatment can include surgery. In some cases, a cancer treatment can include radiation therapy. In some cases, a cancer treatment can include administration of one or more therapeutic agents (e.g., one or more anti-cancer agents). Examples of anti-cancer agents include, without limitation, platinum compounds (e.g., a cisplatin or carboplatin), taxanes (e.g., paclitaxel, docetaxel, or an albumin bound paclitaxel such as nab-paclitaxel), altretamine, capecitabine, cyclophosphamide, etoposide (vp-16), gemcitabine, ifosfamide, irinotecan (cpt-11), liposomal doxorubicin, melphalan, pemetrexed, topotecan, vinorelbine, luteinizing-hormone-releasing hormone (LHRH) agonists (e.g., goserelin and leuprolide), anti-estrogens (e.g., tamoxifen), aromatase inhibitors (e.g., letrozole, anastrozole, and exemestane), angiogenesis inhibitors (e.g., bevacizumab), poly(ADP)-ribose polymerase (PARP) inhibitors (e.g., olaparib, rucaparib, and niraparib), radioactive phosphorus, anti-CTLA-4 antibodies, anti-PD-1 antibodies, anti-PD-L1 antibodies, IL-2 and other cytokines, other bispecific antibodies, and any combinations thereof. In cases where one or more molecules including one or more antigen-binding domains (e.g., scFvs) that can bind to a modified TP53 R175H mutant peptide described herein (e.g., comprising or consisting of SEQ ID NO: 1) are used in combination with one or more additional cancer treatments, the one or more additional cancer treatments can be administered at the same time or independently. For example, a composition including one or more molecules including one or more antigen-binding domains (e.g., scFvs) that can bind to a modified TP53 R175H mutant peptide described herein (e.g., comprising or consisting of SEQ ID NO: 1) can be administered first, and the one or more additional cancer treatments administered second, or vice versa.

Also provided herein are kits that include one or more molecules including one or more antigen-binding domains (e.g., scFvs) that can bind to a modified TP53 R175H mutant peptide described herein (e.g., comprising or consisting of SEQ ID NO: 1). For example, a kit can include a composition (e.g., a pharmaceutically acceptable composition) containing one or more molecules including one or more antigen-binding domains (e.g., scFvs) that can bind to a modified TP53 R175H mutant peptide described herein (e.g., comprising or consisting of SEQ ID NO: 1). In some cases, a kit can include instructions for performing any of the methods described herein. In some cases, a kit can include at least one dose of any of the compositions (e.g., pharmaceutical compositions) described herein. In some cases, a kit can provide a means (e.g., a syringe) for administering any of the compositions (e.g., pharmaceutical compositions) described herein.

The invention will be further described in the following examples, which do not limit the scope of the invention described in the claims.

### EXAMPLES

The invention is further described in the following examples, which do not limit the scope of the invention described in the claims.

### Example 1: Screening Single Mutant Variants for Improved Binding Properties

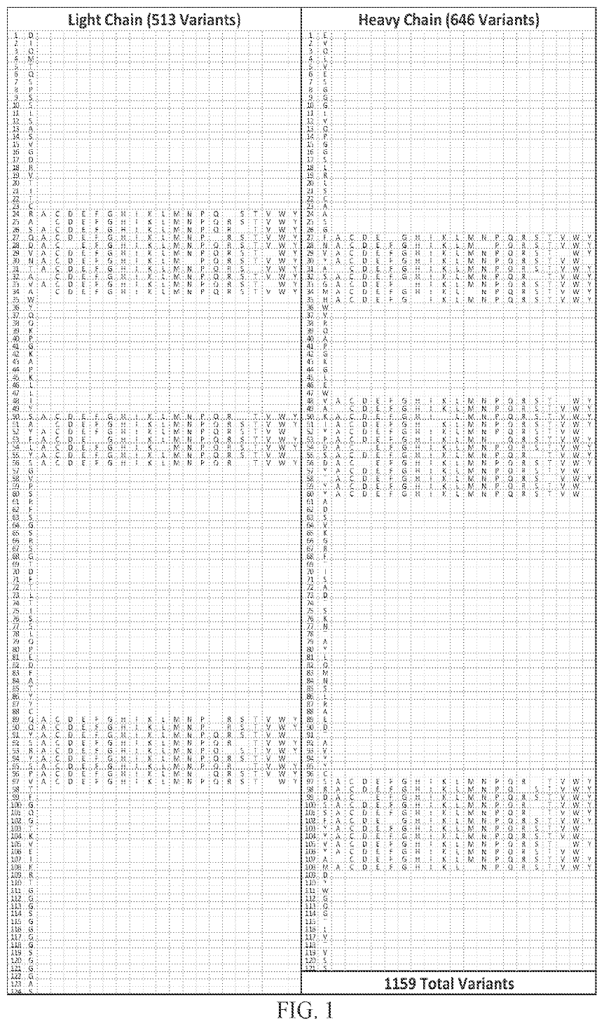

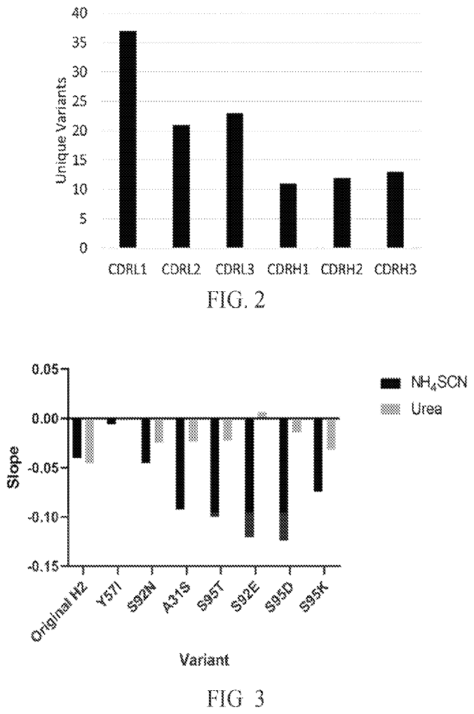

A screen was carried out for improved R175H/HLA-A2 targeting variants using a phage display library consisting of 1159 single chain variable fragments (scFvs) each with a single amino acid change from the original H2 R175H-targeting scFv (SEQ ID NO: 4). A total of 61 sites across the six complementarity determining regions (CDRS) were included in the single mutant library with all 20 amino acids (excluding wild type) represented at each site (FIG. 1). To identify clones with high specificity for the R175H/HLA-A2 pMHC, 5 rounds of panning with negative selection against R175WT pMHC and TP53 R175WT HLA-A2-positive cell lines followed by positive selection for the R175H pMHC was completed. Initial testing indicated that after 5 rounds of panning, phage pools remained diverse with variants present across all six CDRs (FIG. 2).

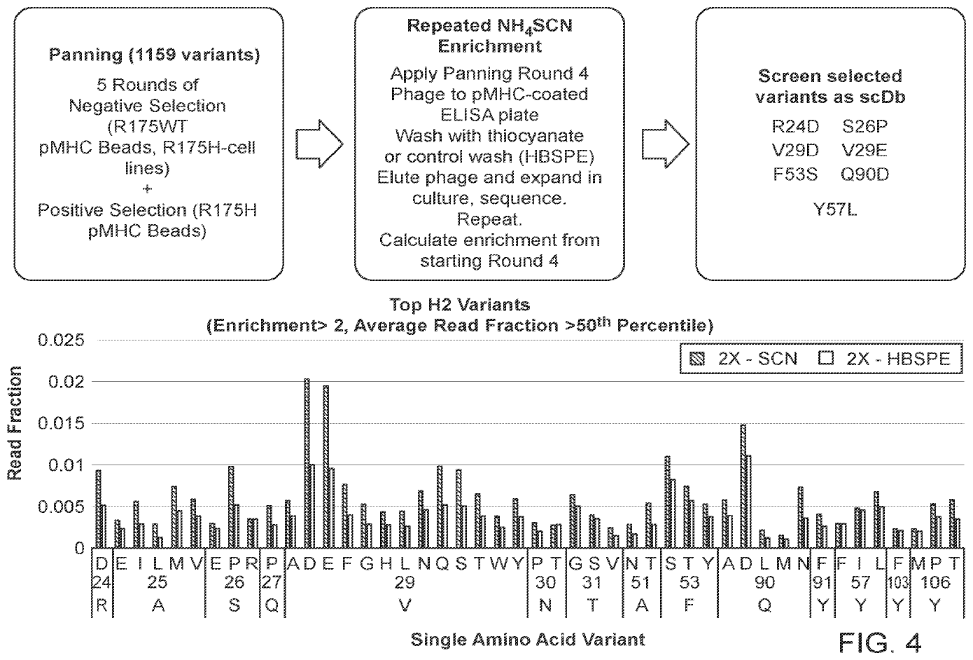

Using data from the crystal structure of the H2 Fab bound to HLA-A2, panning clones with potential structural relevance were selected to screen under more stringent binding conditions. Ammonium thiocyanate (NH₄SCN) and urea washes were used to eliminate scFvs with weak binding to the R175H monomer and identify clones specific for R175H over R175WT (FIG. 3). Additional NH₄SCN-based screening of pooled phage from panning rounds 4 and 5 identified several variants that were enriched with thiocyanate washes (FIG. 4).

### Example 2: SCN-Resistant Variants Have Strong Relative Binding to the R175H/HLA-A2 Monomer

Variants that maintained strong binding to the R175H monomer under stringent wash conditions were then converted into the single-chain diabody (scDb) bispecific antibody format for functional testing. In order, each scDb comprises an IL-2 signal sequence (SEQ ID NO: 6), the pHLA-targeting scFv light chain (mutants of SEQ ID NO: 7), a GGGGS (G4S) linker (SEQ ID NO: 11), anti-CD3 scFv heavy chain (SEQ ID NO: 12), 3×G4S linkers (SEQ ID NO: 13), anti-CD3 scFv light chain (SEQ ID NO: 14), G4S (SEQ ID NO: 15), pHLA-targeting scFv heavy chain (mutants of SEQ ID NO: 16), and a 5×His affinity tag for purification. scDbs were expressed in HEK293FT cells and purified using NiNTA resin.

To confirm that the expressed scDbs bind the R175H/A2 monomer specifically and can bind CD3, scDbs were applied to plates coated with R175H/A2 monomer, R175WT/A2 monomer or the CD3delta/epsilon heterodimer. When normalized to CD3 binding signal, most variant scDbs demonstrated increased relative binding to the R175H/HLA-A2 monomer compared to the original H2-scDb by ELISA (FIG. 5).

### Example 3: Identifying Variants with Improved Sensitivity and Activity Against R175H-HLA-A2 In Vitro

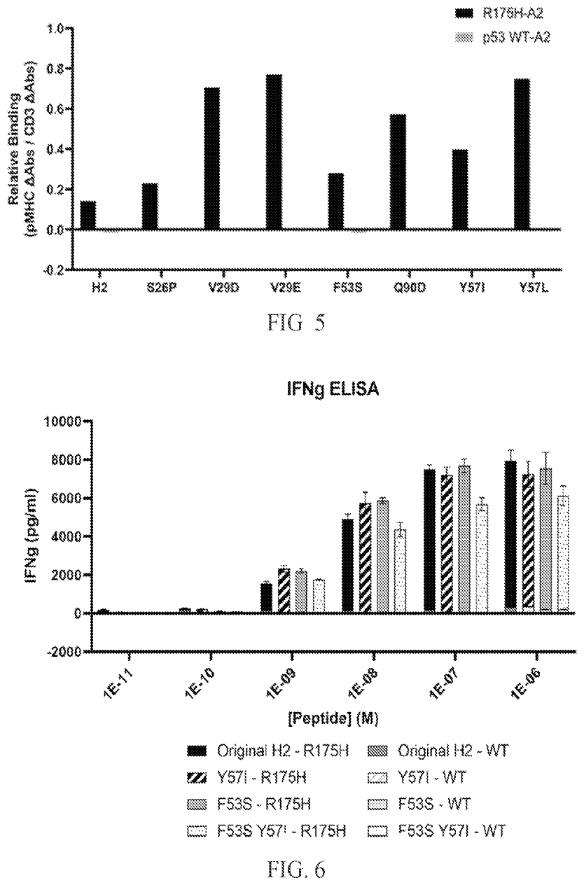

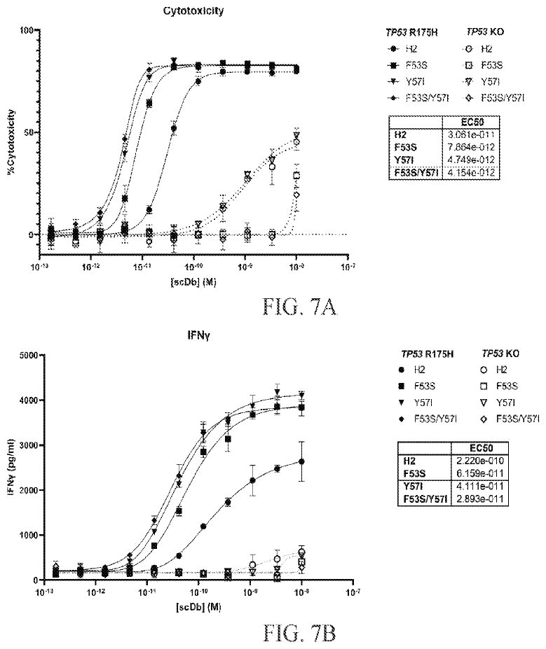

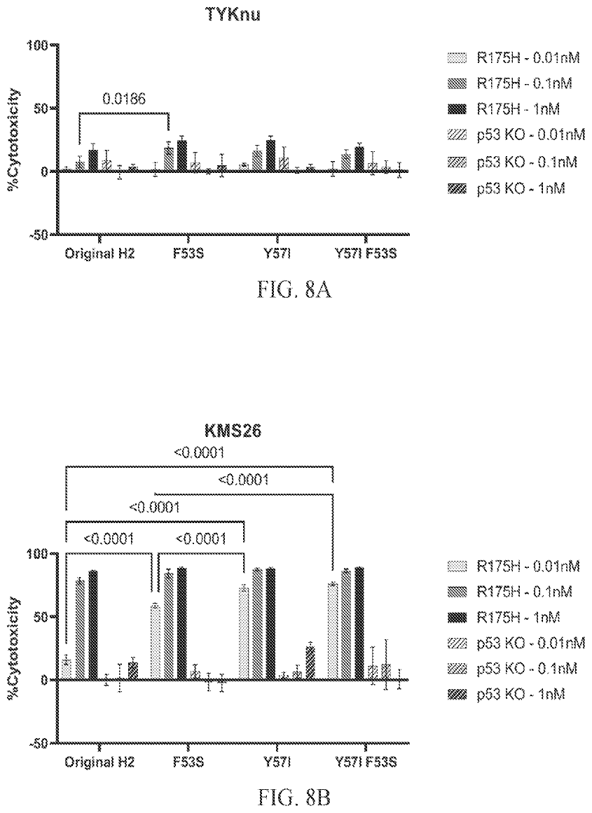

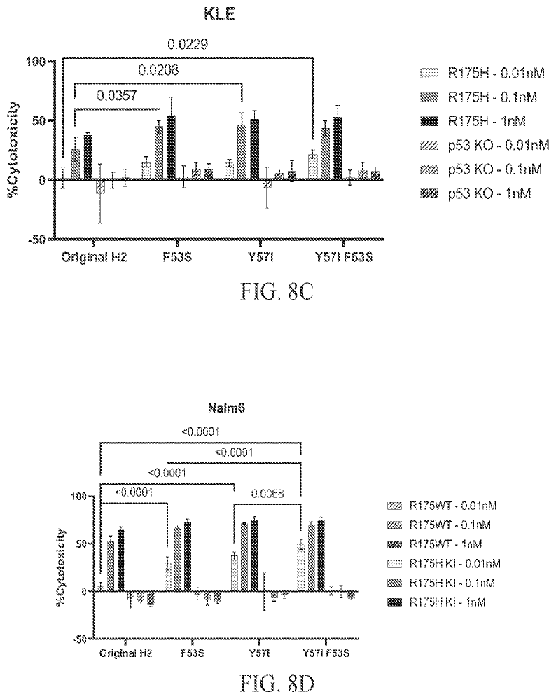

Three candidate variants, including a double mutant combining the two most potent single mutants, (F53S (SEQ ID NO: 21), Y57I (SEQ ID NO: 25), and F53S/Y57I (SEQ ID NO: 29)) underwent functional testing as scDbs in vitro and in vivo. Variant expression plasmids were generated by site directed mutagenesis and used to express each scDb in HEK293FT cells. scDbs for in vivo testing were produced in larger scale and purified by size exclusion chromatography. Variant scDbs were tested for their ability to activate human T cells in co-culture with TAP-deficient T2A3 cells pulsed with varying levels of R175H or R175WT 9mer peptide. Variants Y57I and F53S demonstrated improved sensitivity at low peptide pulsing concentrations (1 nM) compared to the original H2 scDb (FIG. 6). All H2 variants had increased cytotoxicity, interferon gamma response and lower EC₅₀ values than the original H2 scDb in an overnight co-culture with KMS26 cells and human T cells in the presence of varying concentrations of scDb (FIGS. 7A-7B). The cytotoxicity EC₅₀ values for F53S, Y57I and F53S/Y57I against KMS26-p53^{R175H} were 7.864×10⁻¹² M, 4.749×10⁻² M and 4.154×10⁻¹² M compared to 3.061×10⁻¹¹ M for H2 scDb. At low scDb concentrations (13.7 pM and 4.57 pM), an ordinary two-way ANOVA with Tukey's multiple comparisons tests indicates that all H2 variants have increased cytotoxicity compared to H2 scDb (P<0.0001) and that Y57I and F53S/Y57I generated increased interferon gamma compared to H2 scDb (P<0.0001). The H2 variants also have improved activity in co-culture against a variety of HLA-A2-positive cell lines with endogenous TP53 R175H mutations (FIGS. 8A-8D). Indeed, Y57I and F53S/Y57I demonstrates similar or greater activity against the KMS26 cell line at ten-fold lower bispecific antibody concentrations compared to the original H2 scDb.

### Example 4: Variant scDbs Bind the R175H/HLA-A2 Complex with a Higher Affinity

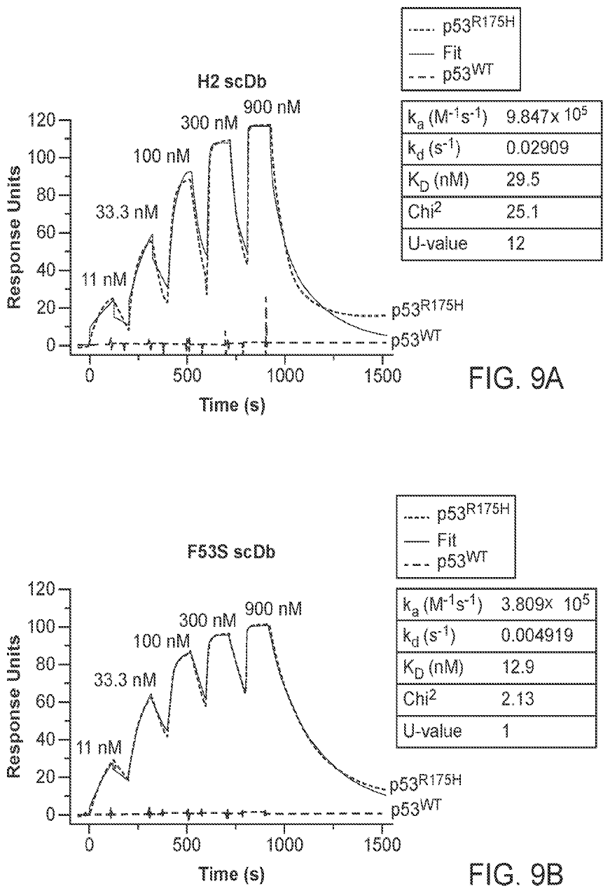

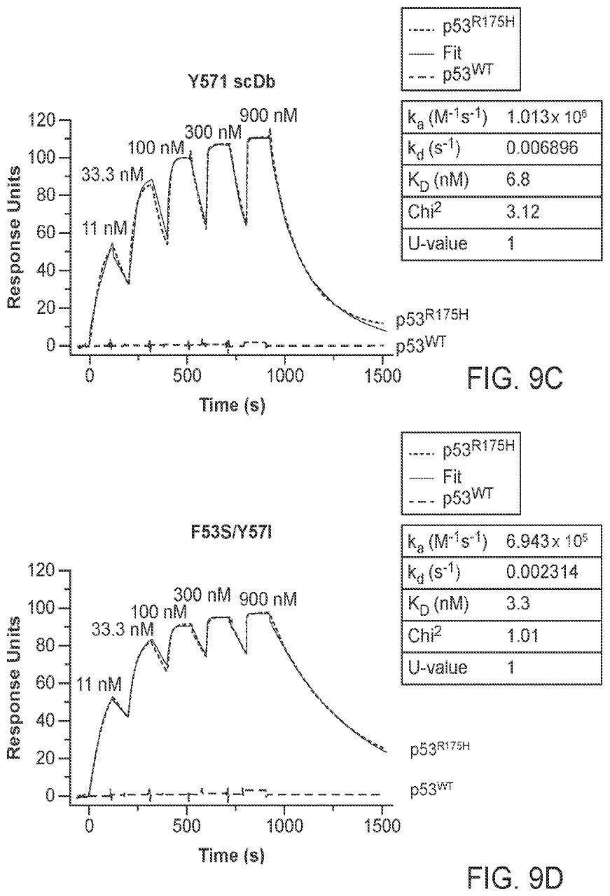

To determine whether the H2 variants bind to the R175H peptide-MHC complex with a different affinity than the original H2 scDb, F53S, Y57I and F53S/Y57I scDb binding to the R175H/A2 monomer was measured by surface plasmon resonance (SPR) (FIGS. 9A-9D). All variants bound to the R175H pMHC complex with higher affinity than the original H2 scDb. The K_{D} for the variants are 12.9 nM, 6.8 nM and 3.3 nM for F53S, Y57I and F53S/Y57I compared to 29.5 nM for the original H2 scDb. Variants did not have increased binding to the p53 WT/A2 pMHC.

### Example 5: Variant scDbs Outperform the Original H2 scDb In Vivo

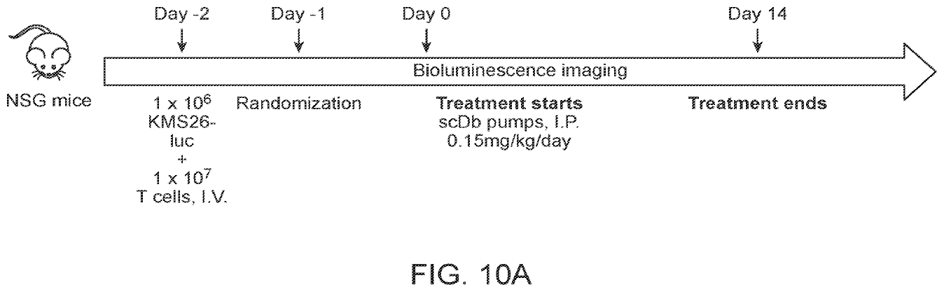

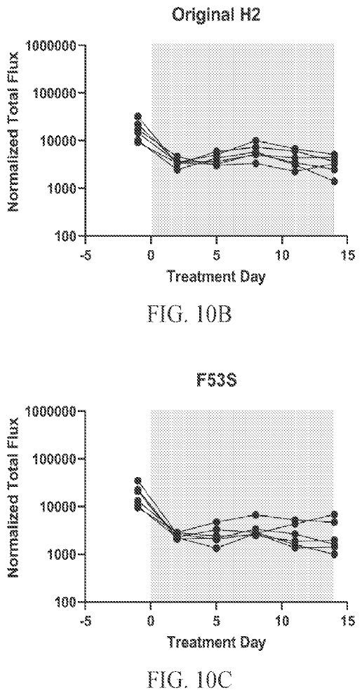

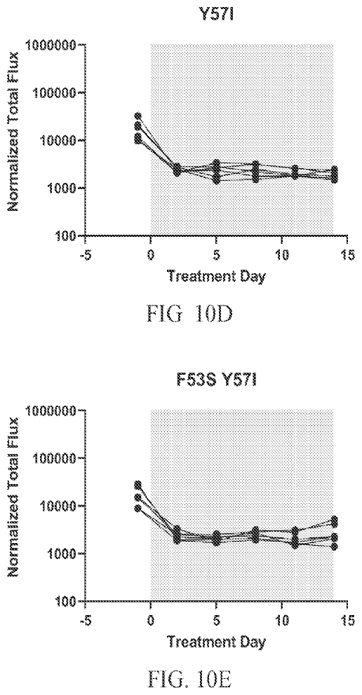

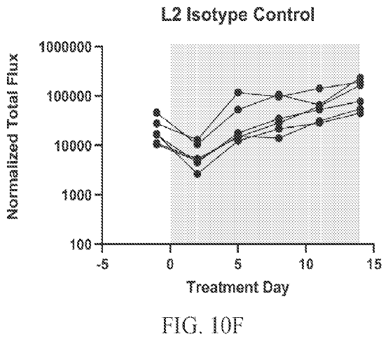

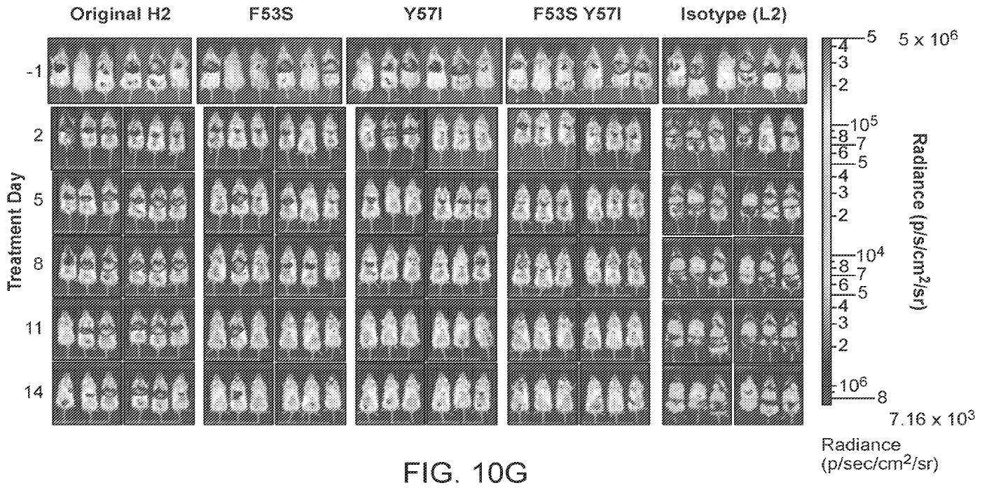

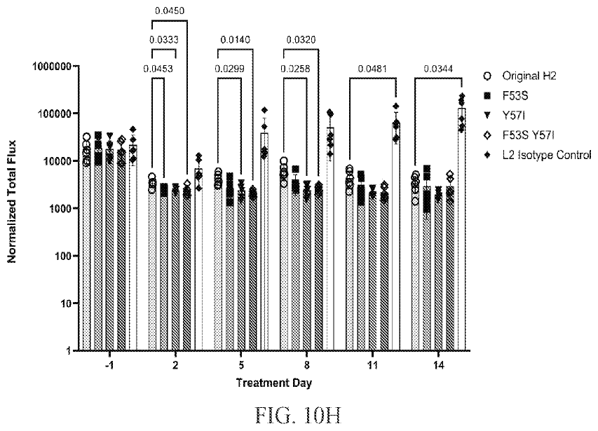

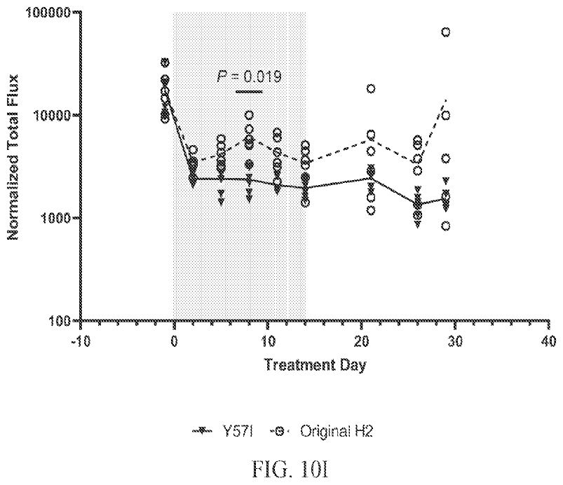

To compare the in vivo anti-tumor efficacy of the 3 H2 variants F53S, Y57I and F53S/Y57I to the original H2 scDb, 13-15 week-old female NSG mice (NOD scid IL2rg) were intravenously inoculated with 1×10⁶ KMS26 cells expressing luciferase (TP53 R175H, HLA-A2) and 1×10⁷ human T cells. Two days later, mice were treated with 0.15 mg/kg/day scDb using a 14-day continuous release pump surgically placed in the mouse peritoneal space (FIG. 10A-10G). N=6 mice per group. Tumor burden was monitored by bioluminescent imaging. Total flux measurements were normalized to an injection control fluorescent dye measured over the thorax for each mouse. FIG. 10H indicates that F53S, Y57I and F53S/Y57I had improved tumor control compared to the original H2 scDb on treatment day 2 and that Y57I and F53S/Y57I had improved tumor control compared to the original H2 scDb on treatment days 5 and 8 (P<0.05, Two-way repeated measures ANOVA with Geissner-Greenhouse correction and Dunnett's multiple comparisons test). FIG. 10I demonstrates that Y57I controlled KMS26 tumor growth in vivo. Multiple Mann-Whitney tests of the bioluminescent data from FIGS. 10A-10G indicate that Y57I had improved tumor control at Day 8 compared to the original H2 scDb (P=0.019). In the two weeks following treatment, no Y57I-treated mice had tumor recurrence.

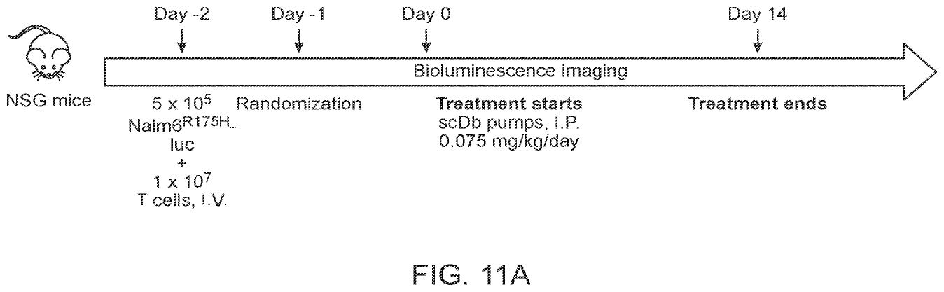

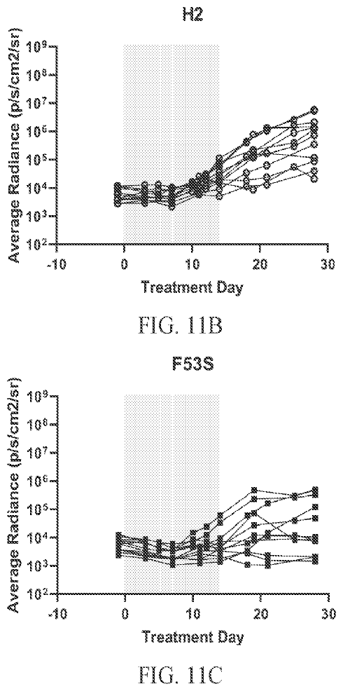

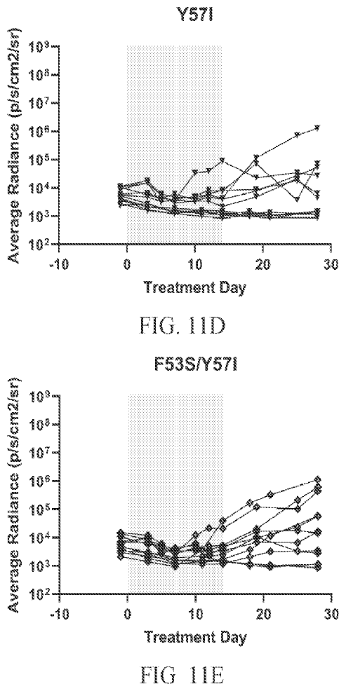

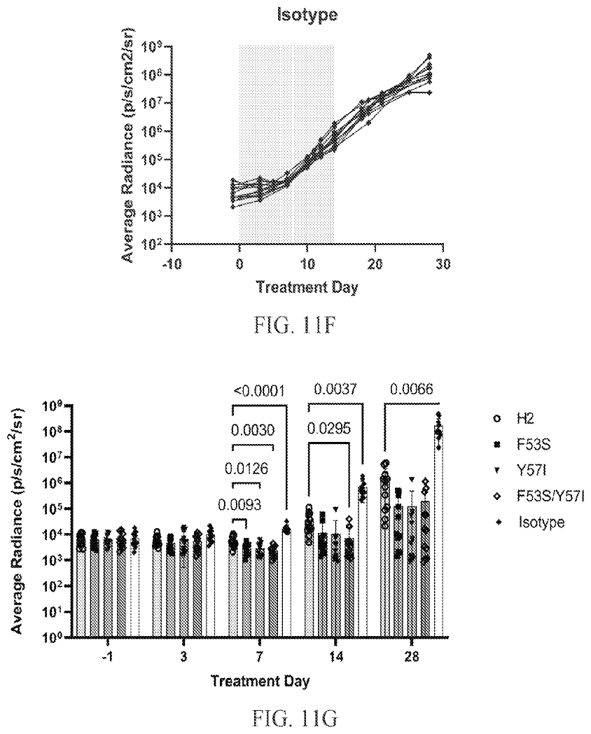

Because the variant scDbs and original H2 had anti-tumor efficacy against KMS26 at a 0.15 mg/kg/d dosing regimen, the variant scDbs were tested at a 50% lower dose for anti-tumor efficacy against a second tumor model with faster growth kinetics-Nalm6. Luciferase-expressing Nalm6 cells were genetically modified by CRISPR to harbor a homozygous p53^{R175H} mutation. 7-8-week old NSG (NOD scid IL2rg) mice were intravenously inoculated with 5×10⁵ Nalm6^{R175H} cells and 1×10⁷ human T cells (FIG. 11A). Two days later, mice were treated with 0.075 mg/kg/d scDb using a 14-day continuous release pump surgically placed in the mouse peritoneal space (FIG. 11B-11). Tumor burden was monitored by bioluminescent imaging. Bioluminescence data from two independent experiments (N=6 per scDb) were combined for a total of N=12 per scDb. At treatment day 7, two-way repeated measures ANOVA with Geissner-Greenhouse correction and Dunnett's multiple comparisons test to H2 scDb indicated that all H2 variants had improved tumor control (H2 vs. F53S P=0.0093, Y57I P=0.0126, F53S/Y57I P=0.0030) and that H2 was superior to the isotype control scDb (P<0.0001). F53S/Y57I had superior tumor control compared to H2 at day 14 by Dunnett's multiple comparisons test (2-way RM ANOVA, P=0.0295).

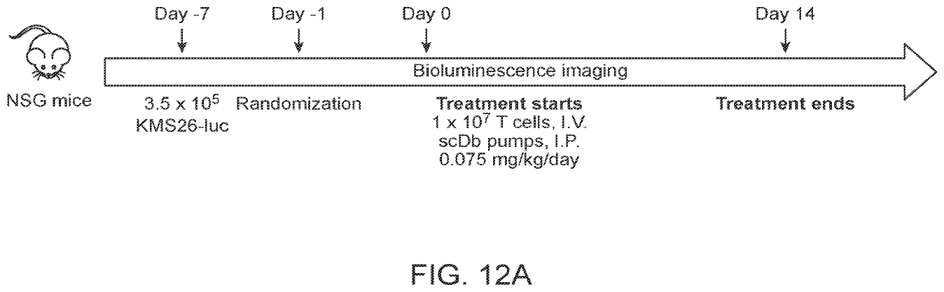

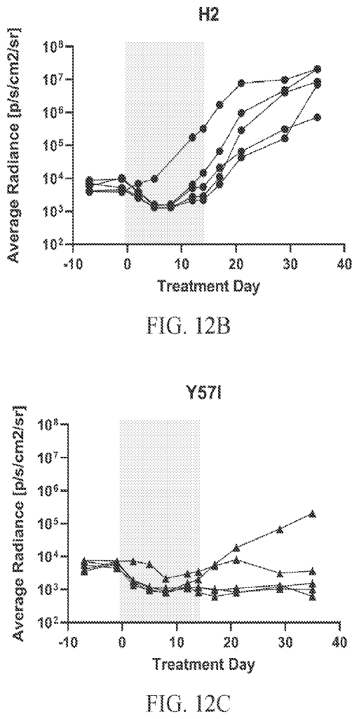

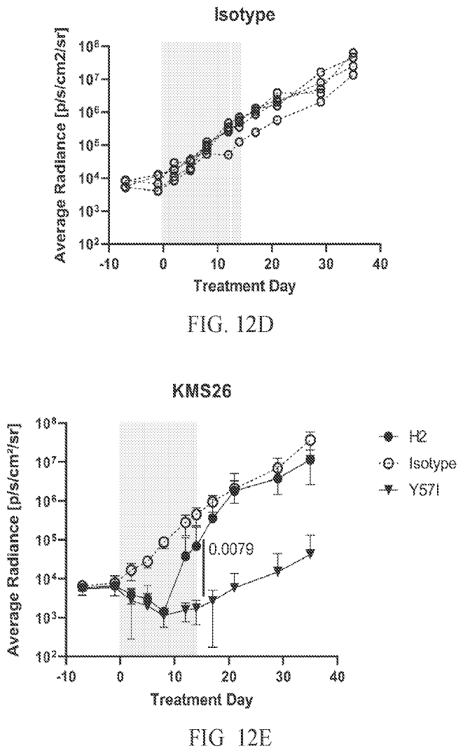

To determine whether Y57I performs better than the original H2 scDb in an established tumor model, Y57I scDb was tested in a delayed treatment model with KMS26 cells. 7-9-week old NSG (NOD scid IL2rg) mice were intravenously inoculated with 3.5×10⁵ luciferase-expressing KMS26 cells. Seven days later, mice were treated with 1×10⁷ human T cells intravenously and 0.075 mg/kg/d scDb using a 14-day continuous release pump surgically placed in the mouse peritoneal space (FIG. 12A-12E). Tumor burden was monitored by bioluminescent imaging. N=5 per group. A Mann-Whitney test indicated that Y57I treated mice had lower tumor burden than mice treated with the original H2 scDb at the end of treatment on day 14 (P 0.0079).

### SEQUENCES

SEQ ID NO: 1 P53 R175H mutant peptide

HMTEVVRHC

SEQ ID NO: 2 P53 R175 wild type peptide

HMTEVVRRC

SEQ ID NO: 3 p53
>sp|P04637|P53_HUMAN Cellular tumor antigen p53 OS=Homo sapiens OX=9606 GN=TP53 PE=1 SV=4

MEEPQSDPSVEPPLSQETFSDLWKLLPENNVLSPLPSQAMDDLMLSPDD
IEQWFTEDPGPDEAPRMPEAAPPVAPAPAAPTPAAPAPAPSWPLSSSVP
SQKTYQGSYGFRLGFLHSGTAKSVTCTYSPALNKMFCQLAKTCPVQLWV
DSTPPPGTRVRAMAIYKQSQHMTEVVRRCPHHERCSDSDGLAPPQHLIR
VEGNLRVEYLDDRNTFRHSVVVPYEPPEVGSDCTTIHYNYMCNSSCMGG
MNRRPILTIITLEDSSGNLLGRNSFEVRVCACPGRDRRTEEENLRKKGE
PHHELPPGSTKRALPNNTSSSPQPKKKPLDGEYFTLQIRGRERFEMFRE
LNEALELKDAQAGKEPGGSRAHSSHLKSKKGOSTSRHKKLMEKTEGPDS
D

SEQ ID NO: 4 H2 scFv

DIQMTQSPSSLSASVGDRVTITCRASQDVNTAVAWYQQKPGKAPKLLIY
SAYFLYSGVPSRFSGSRSGTDFTLTISSLQPEDFATYYCQQYSRYSPVT
FGQGTKVEIKRTGGGSGGGGSGGGASEVQLVESGGGLVQPGGSLRLSCA
ASGFNVYASGMHWVRQAPGKGLEWVAKIYPDSDYTYYADSVKGRFTISA
DTSKNTAYLQMNSLRAEDTAVYYCSRDSSFYYVYAMDYWGQGTLVTVSS

SEQ ID NO: 5 H2 scDb

MYRMQLLSCIALSLALVINSDIQMTQSPSSLSASVGDRVTITCRASQDV
NTAVAWYQQKPGKAPKLLIYSAYFLYSGVPSRFSGSRSGTDFTLTISSL
QPEDFATYYCQQYSRYSPVTFGQGTKVEIKGGGGSEVQLQQSGPELVKP
GASMKISCKASGYSFTGYTMNWVKQSHGKNLEWMGLINPYKGVSTYNQK
FKDKATLTVDKSSSTAYMELLSLTSEDSAVYYCARSGYYGDSDWYFDVW
GAGTTVTVSSGGGGSGGGGSGGGGSDIQMTQTTSSLSASLGDRVTISCR
ASQDIRNYLNWYQQKPDGTVKLLIYYTSRLHSGVPSKFSGSGSGTDYSL
TISNLEQEDIATYFCQQGNTLPWTFAGGTKLEIKEVQLVESGGGLVQPG
GSLRLSCAASGFNVYASGMHWVRQAPGKGLEWVAKIYPDSDYTYYADSV
KGRFTISADTSKNTAYLQMNSLRAEDTAVYYCSRDSSFYYVYAMDYWGQ
GTLVTVSSHHHHHH

SEQ ID NO: 6 H2 IL-2 signal sequence

MYRMQLLSCIALSLALVINS

SEQ ID NO: 7 H2 R175H peptide-targeting scFv Light Chain

DIQMTQSPSSLSASVGDRVTITCRASQDVNTAVAWYQQKPGKAPKLLIY
SAYFLYSGVPSRFSGSRSGTDFTLTISSLQPEDFATYYCQQYSRYSPVT
FGQGTKVEIK

SEQ ID NO: 8 H2 R175H peptide-targeting scFv Light Chain CDR1

RASQDVNTAVA

SEQ ID NO: 9 H2 R175H peptide-targeting scFv Light Chain CDR2

SAYFLYS

SEQ ID NO: 10 H2 R175H peptide-targeting scFv Light Chain CDR3

QQYSRYSPV

SEQ ID NO: 11 Linker

GGGGS

SEQ ID NO: 12 H2 CD3-targeting Heavy Chain

EVQLQQSGPELVKPGASMKISCKASGYSFTGYTMNWVKQSHGKNLEWMG
LINPYKGVSTYNQKFKDKATLTVDKSSSTAYMELLSLTSEDSAVYYCAR
SGYYGDSDWYFDVWGAGTTVTVSS

SEQ ID NO: 13 Linker

GGGGSGGGGSGGGGS

SEQ ID NO: 14 H2 CD3-targeting Light Chain

DIQMTQTTSSLSASLGDRVTISCRASQDIRNYLNWYQQKPDGTVKLLIY
YTSRLHSGVPSKFSGSGSGTDYSLTISNLEQEDIATYFCQQGNTLPWTF
AGGTKLEIK

SEQ ID NO: 15 Linker

GGGGS

SEQ ID NO: 16 H2 R175H peptide-targeting scFv Heavy Chain

EVQLVESGGGLVQPGGSLRLSCAASGFNVYASGMHWVRQAPGKGLEWVA
KIYPDSDYTYYADSVKGRFTISADTSKNTAYLQMNSLRAEDTAVYYCSR
DSSFYYVYAMDYWGQGTLVTVSS

SEQ ID NO: 17 H2 R175H peptide-targeting scFv Heavy Chain CDRH1

FNVYASGMH

SEQ ID NO: 18 H2 R175H peptide-targeting scFv Heavy Chain CDRH2

VAKIYPDSDYTYY

SEQ ID NO: 19 H2 R175H peptide-targeting scFv Heavy Chain CDRH3

SRDSSFYYVYAM

SEQ ID NO: 20 F53S scFv

DIQMTQSPSSLSASVGDRVTITCRASQDVNTAVAWYQQKPGKAPKLLIY
SAYSLYSGVPSRFSGSRSGTDFTLTISSLQPEDFATYYCQQYSRYSPVT
FGQGTKVEIKRTGGGSGGGGSGGGASEVQLVESGGGLVQPGGSLRLSCA
ASGFNVYASGMHWVRQAPGKGLEWVAKIYPDSDYTYYADSVKGRFTISA
DTSKNTAYLQMNSLRAEDTAVYYCSRDSSFYYVYAMDYWGQGTLVTVSS

SEQ ID NO: 21 F53S R175H peptide-targeting scDb (without signal sequence or His tag)

MYRMQLLSCIALSLALVINSDIQMTQSPSSLSASVGDRVTITCRASQDV
NTAVAWYQQKPGKAPKLLIYSAYSLYSGVPSRFSGSRSGTDFTLTISSL
QPEDFATYYCQQYSRYSPVTFGQGTKVEIKGGGGSEVQLQQSGPELVKP
GASMKISCKASGYSFTGYTMNWVKQSHGKNLEWMGLINPYKGVSTYNQK
FKDKATLTVDKSSSTAYMELLSLTSEDSAVYYCARSGYYGDSDWYFDVW
GAGTTVTVSSGGGGSGGGGSGGGGSDIQMTQTTSSLSASLGDRVTISCR
ASQDIRNYLNWYQQKPDGTVKLLIYYTSRLHSGVPSKESGSGSGTDYSL
TISNLEQEDIATYFCQQGNTLPWTFAGGTKLEIKGGGGSEVQLVESGGG
LVQPGGSLRLSCAASGFNVYASGMHWVRQAPGKGLEWVAKIYPDSDYTY
YADSVKGRFTISADTSKNTAYLQMNSLRAEDTAVYYCSRDSSFYYVYAM
DYWGQGTLVTVSS

SEQ ID NO: 22 F53S R175H peptide-targeting scFv Light Chain

DIQMTQSPSSLSASVGDRVTITCRASQDVNTAVAWYQQKPGKAPKLLIY
SAYSLYSGVPSRFSGSRSGTDFTLTISSLQPEDFATYYCQQYSRYSPVT
FGQGTKVEIK

SEQ ID NO: 23 F53S R175H peptide-targeting scFv Light Chain CDR2

SAYSLYS

SEQ ID NO: 24 Y57I scFv

DIQMTQSPSSLSASVGDRVTITCRASQDVNTAVAWYQQKPGKAPKLLIY
SAYFLYSGVPSRFSGSRSGTDFTLTISSLQPEDFATYYCQQYSRYSPVT
FGQGTKVEIKRTGGGSGGGGSGGGASEVQLVESGGGLVQPGGSLRLSCA
ASGFNVYASGMHWVRQAPGKGLEWVAKIYPDSDITYYADSVKGRFTISA
DTSKNTAYLQMNSLRAEDTAVYYCSRDSSFYYVYAMDYWGQGTLVTVSS

SEQ ID NO: 25 Y57I R175H peptide-targeting scDb (without signal sequence or His tag)

MYRMQLLSCIALSLALVTNSDIQMTQSPSSLSASVGDRVTITCRASQDV
NTAVAWYQQKPGKAPKLLIYSAYFLYSGVPSRFSGSRSGTDFTLTISSL
QPEDFATYYCQQYSRYSPVTFGQGTKVEIKGGGGSEVQLQQSGPELVKP
GASMKISCKASGYSFTGYTMNWVKQSHGKNLEWMGLINPYKGVSTYNQK
FKDKATLTVDKSSSTAYMELLSLTSEDSAVYYCARSGYYGDSDWYFDVW
GAGTTVTVSSGGGGSGGGGSGGGGSDIQMTQTTSSLSASLGDRVTISCR
ASQDIRNYLNWYQQKPDGTVKLLIYYTSRLHSGVPSKESGSGSGTDYSL
TISNLEQEDIATYFCQQGNTLPWTFAGGTKLEIKGGGGSEVQLVESGGG
LVQPGGSLRLSCAASGFNVYASGMHWVRQAPGKGLEWVAKIYPDSDITY
YADSVKGRFTISADTSKNTAYLQMNSLRAEDTAVYYCSRDSSFYYVYAM
DYWGQGTLVTVSS

SEQ ID NO: 26 Y57I R175H peptide-targeting scFv Heavy Chain

EVQLVESGGGLVQPGGSLRLSCAASGFNVYASGMHWVRQAPGKGLEWVA
KIYPDSDITYYADSVKGRFTISADTSKNTAYLQMNSLRAEDTAVYYCSR
DSSFYYVYAMDYWGQGTLVTVSS

SEQ ID NO: 27 Y57I R175H peptide-targeting scFv Heavy Chain CDR2

VAKIYPDSDITYY

SEQ ID NO: 28 F53S/Y57I scFv

DIQMTQSPSSLSASVGDRVTITCRASQDVNTAVAWYQQKPGKAPKLLIY
SAYSLYSGVPSRFSGSRSGTDFTLTISSLQPEDFATYYCQQYSRYSPVT
FGQGTKVEIKRTGGGSGGGGSGGGASEVQLVESGGGLVQPGGSLRLSCA
ASGFNVYASGMHWVRQAPGKGLEWVAKIYPDSDITYYADSVKGRFTISA
DTSKNTAYLQMNSLRAEDTAVYYCSRDSSFYYVYAMDYWGQGTLVTVS

SEQ ID NO: 29 F53S/Y57I R175H peptide-targeting scDb (without signal sequence or His tag)

MYRMQLLSCIALSLALVINSDIQMTQSPSSLSASVGDRVTITCRASQDV
NTAVAWYQQKPGKAPKLLIYSAYSLYSGVPSRFSGSRSGTDFTLTISSL
QPEDFATYYCQQYSRYSPVTFGQGTKVEIKGGGGSEVQLQQSGPELVKP
GASMKISCKASGYSFTGYTMNWVKQSHGKNLEWMGLINPYKGVSTYNQK
FKDKATLTVDKSSSTAYMELLSLTSEDSAVYYCARSGYYGDSDWYFDVW
GAGTTVTVSSGGGGSGGGGSGGGGSDIQMTQTTSSLSASLGDRVTISCR
ASQDIRNYLNWYQQKPDGTVKLLIYYTSRLHSGVPSKFSGSGSGTDYSL
TISNLEQEDIATYFCQQGNTLPWTFAGGTKLEIKGGGGSEVQLVESGGG
LVQPGGSLRLSCAASGENVYASGMHWVRQAPGKGLEWVAKIYPDSDITY
YADSVKGRFTISADTSKNTAYLQMNSLRAEDTAVYYCSRDSSFYYVYAM
DYWGQGTLVTVSS

TABLE 1
R175H peptide-targeting scFvs

| scFv | Variable light | SEQ ID | Variable heavy | SEQ ID |
|---|---|---|---|---|
| name | chain (AA) | NO: | chain (AA) | NO: |
| H2 | DIQMTQSPSSLSASVGDR | SEQ ID | EVQLVESGGGLVQPGGSLR | SEQ ID |
| | VTITCRASQDVNTAVAWY | NO: 30 | LSCAASGFNVYASGMHWVR | NO: 31 |
| | QQKPGKAPKLLIYSAYFL | | QAPGKGLEWVAKIYPDSDY | |
| | YSGVPSRFSGSRSGTDFT | | TYYADSVKGRFTISADTSK | |
| | LTISSLQPEDFATYYCQQ | | NTAYLQMNSLRAEDTAVYY | |
| | YSRYSPVTFGQGTKVEIK | | CSRDSSFYYVYAMDYWGQG | |
| | | | TLVTVSS | |
| F53S | DIQMTQSPSSLSASVGDR | SEQ ID | EVQLVESGGGLVQPGGSLR | SEQ ID |
| | VTITCRASQDVNTAVAWY | NO: 32 | LSCAASGFNVYASGMHWVR | NO: 33 |
| | QQKPGKAPKLLIYSAYSL | | QAPGKGLEWVAKIYPDSDY | |
| | YSGVPSRFSGSRSGTDFT | | TYYADSVKGRFTISADTSK | |
| | LTISSLQPEDFATYYCQQ | | NTAYLQMNSLRAEDTAVYY | |
| | YSRYSPVTFGQGTKVEIK | | CSRDSSFYYVYAMDYWGQG | |
| | | | TLVTVSS | |
| Y57I | DIQMTQSPSSLSASVGDR | SEQ ID | EVQLVESGGGLVQPGGSLR | SEQ ID |
| | VTITCRASQDVNTAVAWY | NO: 34 | LSCAASGFNVYASGMHWVR | NO: 35 |
| | QQKPGKAPKLLIYSAYFL | | QAPGKGLEWVAKIYPDSDI | |
| | YSGVPSRFSGSRSGTDFT | | TYYADSVKGRFTISADTSK | |
| | LTISSLQPEDFATYYCQQ | | NTAYLQMNSLRAEDTAVYY | |
| | YSRYSPVTFGQGTKVEIK | | CSRDSSFYYVYAMDYWGQG | |
| | | | TLVTVSS | |
| F35S/ | DIQMTQSPSSLSASVGDR | SEQ ID | EVQLVESGGGLVQPGGSLR | SEQ ID |
| Y57I | VTITCRASQDVNTAVAWY | NO: 36 | LSCAASGFNVYASGMHWVR | NO: 37 |
| | QQKPGKAPKLLIYSAYSL | | QAPGKGLEWVAKIYPDSDI | |
| | YSGVPSRFSGSRSGTDFT | | TYYADSVKGRFTISADTSK | |
| | LTISSLQPEDFATYYCQQ | | NTAYLQMNSLRAEDTAVYY | |
| | YSRYSPVTFGQGTKVEIK | | CSRDSSFYYVYAMDYWGQG | |
| | | | TLVTVSS | |

TABLE 2
Anti-human CD3 scFv sequences

| Clone Name and | |
|---|---|
| SEQ ID NO: | Clone scFv Sequence |
| SEQ ID NO: 38 | DIQMTQSPSSLSASVGDRVTITCRASQDIRNYLNWYQQKPGKAPKLLIYYTSR |
| humanized UCHT1 | LESGVPSRFSGSGSGTDYTLTISSLQPEDFATYYCQQGNTLPWTFGQGTKVEI |
| (hUCHT1v9) | KGGGGSGGGGSGGGGSEVQLVESGGGLVQPGGSLRLSCAASGYSFTGYTMNWV |
| | RQAPGKGLEWVALINPYKGVSTYNQKFKDRFTISVDKSKNTAYLQMNSLRAED |
| | TAVYYCARSGYYGDSDWYFDVWGQGTLVTVSS |
| SEQ ID NO: 39 | DIQMTQTTSSLSASLGDRVTISCRASQDIRNYLNWYQQKPDGTVKLLIYYTSR |
| murine UCHT1 | LHSGVPSKFSGSGSGTDYSLTISNLEQEDIATYFCQQGNTLPWTFAGGTKLEI |
| (mUCHT1) | KGGGGSGGGGSGGGGSEVQLQQSGPELVKPGASMKISCKASGYSFTGYTMNWV |
| | KQSHGKNLEWMGLINPYKGVSTYNQKFKDKATLTVDKSSSTAYMELLSLTSED |
| | SAVYYCARSGYYGDSDWYFDVWGAGTTVTVSS |
| SEQ ID NO: 40 | DIVLTQSPATLSLSPGERATLSCRASQSVSYMNWYQQKPGKAPKRWIYDTSKV |
| diL2K | ASGVPARFSGSGSGTDYSLTINSLEAEDAATYYCQQWSSNPLTFGGGTKVEIK |
| | GGGGSGGGGSGGGGSDVQLVQSGAEVKKPGASVKVSCKASGYTFTRYTMHWVR |
| | QAPGQGLEWIGYINPSRGYTNYADSVKGRFTITTDKSTSTAYMELSSLRSEDT |
| | ATYYCARYYDDHYCLDYWGQGTTVTVSS |
| SEQ ID NO: 41 | QAVVTQEPSLTVSPGGTVTLTCRSSTGAVTTSNYANWVQQKPGQAPRGLIGGT |
| hXR32 | NKRAPWTPARFSGSLLGGKAALTITGAQAEDEADYYCALWYSNLWVFGGGTKL |
| | TVLGGGGSGGGGSGGGGSEVQLVESGGGLVQPGGSLRLSCAASGFTENTYAMN |
| | WVRQAPGKGLEWVARIRSKYNNYATYYADSVKDRFTISRDDSKNSLYLQMNSL |
| | KTEDTAVYYCVRHGNFGNSYVSWFAYWGQGTLVTVSS |
| SEQ ID NO: 42 | DIQLTQSPAIMSASPGEKVTMTCRASSSVSYMNWYQQKSGTSPKRWIYDTSKV |
| L2K-07/TR66 | ASGVPYRFSGSGSGTSYSLTISSMEAEDAATYYCQQWSSNPLTFGAGTKLELK |
| | GGGGSGGGGSGGGGSDIKLQQSGAELARPGASVKMSCKTSGYTFTRYTMHWVK |
| | QRPGQGLEWIGYINPSRGYTNYNQKFKDKATLTTDKSSSTAYMQLSSLTSEDS |
| | AVYYCARYYDDHYCLDYWGQGTTLTVSS |
| SEQ ID NO: 43 | QIVLTQSPAIMSASPGEKVTMTCSASSSVSYMNWYQQKSGTSPKRWIYDTSKL |
| OKT3 | ASGVPAHFRGSGSGTSYSLTISGMEAEDAATYYCQQWSSNPFTFGSGTKLEIN |
| | GGGGSGGGGSGGGGSQVQLQQSGAELARPGASVKMSCKASGYTFTRYTMHWVK |
| | QRPGQGLEWIGYINPSRGYTNYNQKFKDKATLTTDKSSSTAYMQLSSLTSEDS |
| | AVYYCARYYDDHYCLDYWGQGTTLTVSS |
| SEQ ID NO: 44 | QTVVTQEPSLTVSPGGTVTLTCGSSTGAVTSGNYPNWVQQKPGQAPRGLIGGT |
| PSMA-CD3 | KFLAPGTPARFSGSLLGGKAALTLSGVQPEDEAEYYCVLWYSNRWVFGGGTKL |
| | TVLGGGGSGGGGSGGGGSEVQLVESGGGLVQPGGSLKLSCAASGFTENKYAMN |
| | WVRQAPGKGLEWVARIRSKYNNYATYYADSVKDRFTISRDDSKNTAYLQMNNL |
| | KTEDTAVYYCVRHGNFGNSYISYWAYWGQGTLVTVSS |
| SEQ ID NO: 45 | EIVLTQSPATLSLSPGERATLSCRASQSVSSYLAWYQQKPGQAPRLLIYDASN |
| 28F11 | RATGIPARFSGSGSGTDFTLTISSLEPEDFAVYYCQQRSNWPPLTFGGGTKVE |
| | IKGGGGSGGGGSGGGGSQVQLVESGGGVVQPGRSLRLSCAASGFKFSGYGMHW |
| | VRQAPGKGLEWVAVIWYDGSKKYYVDSVKGRFTISRDNSKNTLYLQMNSLRAE |
| | DTAVYYCARQMGYWHFDLWGRGTLVTVSS |
| SEQ ID NO: 46 | EIVLTQSPRTLSLSPGERATLSCRASQSVSSSYLAWYQQKPGQAPRLLIYGAS |
| 27H5-VL1 | SRATGIPDRFSGSGSGTDFTLTISRLDPEDFAVYYCQQYGSSPITFGQGTRLE |
| | IKGGGGSGGGGSGGGGSQVQLVESGGGVVQPGRSLRLSCAASGFTFRSYGMHW |
| | VRQAPGKGLEWVAIIWYDGSKKNYADSVKGRFTISRDNSKNTLYLQMNSLRAE |
| | DTAVYYCARGTGYNWEDPWGQGTLVTVSS |
| SEQ ID NO: 47 | DILMTQSPSSLSASVGDRVTITCRASQGISSALAWYQQKPGKAPKLLIYYASS |
| 27H5-VL2 | LQSGVPSRFSGSGSGTDYTLTISSLQPEDFATYYCQQYYSTLTFGGGTKVEIK |
| | GGGGSGGGGSGGGGSQVQLVESGGGVVQPGRSLRLSCAASGFTFRSYGMHWVR |
| | QAPGKGLEWVAIIWYDGSKKNYADSVKGRFTISRDNSKNTLYLQMNSLRAEDT |
| | AVYYCARGTGYNWFDPWGQGTLVTVSS |
| SEQ ID NO: 48 | EIVLTQSPATLSLSPGERATLSCRASQSVSSYLAWYQQKPGQAPRLLIYDASN |
| 23F10 | RATGIPARFSGSGSGTDFTLTISSLEPEDFAVYYCQQRSNWPPLTFGGGTKVE |
| | IKGGGGSGGGGSGGGGSQVQLVQSGGGVVQSGRSLRLSCAASGFKFSGYGMHW |
| | VRQAPGKGLEWVAVIWYDGSKKYYVDSVKGRFTISRDNSKNTLYLQMNSLRGE |
| | DTAVYYCARQMGYWHFDLWGRGTLVTVSS |
| SEQ ID NO: 49 | EIVLTQSPATLSLSPGERATLSCRASQSVSSYLAWYQQKPGQAPRLLIYDASN |
| 15C3-VL1 | RATGIPARFSGSGSGTDFTLTISSLEPEDFAVYYCQQRSNWPWTFGQGTKVEI |
| | KGGGGSGGGGSGGGGSQVQLVQSGGGVVQPGRSLRLSCVASGFTFSSYGMHWV |
| | RQAPGKGLEWVAAIWYNGRKQDYADSVKGRFTISRDNSKNTLYLQMNSLRAED |
| | TAVYYCTRGTGYNWEDPWGQGTLVTVSS |
| SEQ ID NO: 50 | AIQLTQSPSSLSASVGDRVTITCRASQGISSALAWYQQKPGKAPKLLIYDASS |
| 15C3-VL2 | LESGVPSRFSGSGSGTDFTLTISSLQPEDFATYYCQQFNSYPITFGQGTRLEI |
| | KGGGGSGGGGSGGGGSQVQLVQSGGGVVQPGRSLRLSCVASGFTFSSYGMHWV |
| | RQAPGKGLEWVAAIWYNGRKQDYADSVKGRFTISRDNSKNTLYLQMNSLRAED |
| | TAVYYCTRGTGYNWEDPWGQGTLVTVSS |
| SEQ ID NO: 51 | QIVLSQSPAILSASPGEKVTMTCRASSSVSYMHWYQQKPGSSPKPWIYATSNL |
| hu12F6 | ASGVPARFSGSGSGTSYSLTISRVEAEDAATYYCQQWSSNPPTFGGGTKLETK |
| | RGGGGSGGGGSGGGGSQVQLQQSGAELARPGASVKMSCKASGYTFTSYTMHWV |
| | KQRPGQGLEWIGYINPSSGYTKYNQKFKDKATLTADKSSSTAYMQLSSLTSED |
| | SAVYYCARWQDYDVYFDYWGQGTTLTVSS |

TABLE 3
Anti-human CD3 scFv sequences. Affinities of select anti-CD3 clones
were gathered from the literature.

| | | | | SEQ | | SEQ |
|---|---|---|---|---|---|---|
| scFv | | Affinity | variable | ID | variable | ID |
| name | antigen | (nM) | light chain (AA) | NO | heavy chain (AA) | NO |
| UCHT1 | human | 1.4-2.2 | DIQMTQTTSSLSASLGDRV | SEQ | EVQLQQSGPELVKPGASMKI | SEQ |
| ″U″ | CD3 | | TISCRASQDIRNYLNWYQQ | ID | SCKASGYSFTGYTMNWVKQS | ID |
| | | | KPDGTVKLLIYYTSRLHSG | NO: | HGKNLEWMGLINPYKGVSTY | NO: |
| | | | VPSKFSGSGSGTDYSLTIS | 52 | NQKFKDKATLTVDKSSSTAY | 53 |
| | | | NLEQEDIATYFCQQGNTLP | | MELLSLTSEDSAVYYCARSG | |
| | | | WTFAGGTKLEIK | | YYGDSDWYFDVWGAGTTVTV | |
| | | | | | SS | |
| UCHT1.v9 | human | 2.5-4.7 | DIQMTQSPSSLSASVGDRV | SEQ | EVQLVESGGGLVQPGGSLRL | SEQ |
| ″U2″ | CD3 | | TITCRASQDIRNYLNWYQQ | ID | SCAASGYSFTGYTMNWVRQA | ID |
| | | | KPGKAPKLLIYYTSRLESG | NO: | PGKGLEWVALINPYKGVSTY | NO: |
| | | | VPSRFSGSGSGTDYTLTIS | 54 | NQKFKDRFTISVDKSKNTAY | 55 |
| | | | SLQPEDFATYYCQQGNTLP | | LQMNSLRAEDTAVYYCARSG | |
| | | | WTFGQGTKVEIK | | YYGDSDWYFDVWGQGTLVTV | |
| | | | | | SS | |
| diL2K | human | 87 | DIVLTQSPATLSLSPGERA | SEQ | DVQLVQSGAEVKKPGASVKV | SEQ |
| | CD3 | | TLSCRASQSVSYMNWYQQK | ID | SCKASGYTFTRYTMHWVRQA | ID |
| | | | PGKAPKRWIYDTSKVASGV | NO: | PGQGLEWIGYINPSRGYTNY | NO: |
| | | | PARFSGSGSGTDYSLTINS | 56 | ADSVKGRFTITTDKSTSTAY | 57 |
| | | | LEAEDAATYYCQQWSSNPL | | MELSSLRSEDTATYYCARYY | |
| | | | TFGGGTKVEIK | | DDHYCLDYWGQGTTVTVSS | |
| hXR32 | human | 3.6-6.1 | QAVVTQEPSLTVSPGGTVT | SEQ | EVQLVESGGGLVQPGGSLRL | SEQ |
| | CD3 | | LTCRSSTGAVTTSNYANWV | ID | SCAASGFTFNTYAMNWVRQA | ID |
| | | | QQKPGQAPRGLIGGTNKRA | NO: | PGKGLEWVARIRSKYNNYAT | NO: |
| | | | PWTPARFSGSLLGGKAALT | 58 | YYADSVKDRFTISRDDSKNS | 59 |
| | | | ITGAQAEDEADYYCALWYS | | LYLQMNSLKTEDTAVYYCVR | |
| | | | NLWVFGGGTKLTVL | | HGNFGNSYVSWFAYWGQGTL | |
| | | | | | VTVSS | |
| L2K | human | — | DIQLTQSPAIMSASPGEKV | SEQ | DIKLQQSGAELARPGASVKM | SEQ |
| | CD3 | | TMTCRASSSVSYMNWYQQK | ID | SCKTSGYTFTRYTMHWVKQR | ID |
| | | | SGTSPKRWIYDTSKVASGV | NO: | PGQGLEWIGYINPSRGYTNY | NO: |
| | | | PYRFSGSGSGTSYSLTISS | 60 | NQKFKDKATLTTDKSSSTAY | 61 |
| | | | MEAEDAATYYCQQWSSNPL | | MQLSSLTSEDSAVYYCARYY | |
| | | | TFGAGTKLELK | | DDHYCLDYWGQGTTLTVSS | |
| OKT3 | human | 1.2-1.5 | QIVLTQSPAIMSASPGEKV | SEQ | QVQLQQSGAELARPGASVKM | SEQ |
| | CD3 | | TMTCSASSSVSYMNWYQQK | ID | SCKASGYTFTRYTMHWVKQR | ID |
| | | | SGTSPKRWIYDTSKLASGV | NO: | PGQGLEWIGYINPSRGYTNY | NO: |
| | | | PAHFRGSGSGTSYSLTISG | 62 | NQKFKDKATLTTDKSSSTAY | 63 |
| | | | MEAEDAATYYCQQWSSNPF | | MQLSSLTSEDSAVYYCARYY | |
| | | | TFGSGTKLEIN | | DDHYCLDYWGQGTTLTVSS | |
| 28F11 | human | — | EIVLTQSPATLSLSPGERA | SEQ | QVQLVESGGGVVQPGRSLRL | SEQ |
| | CD3 | | TLSCRASQSVSSYLAWYQQ | ID | SCAASGFKFSGYGMHWVRQA | ID |
| | | | KPGQAPRLLIYDASNRATG | NO: | PGKGLEWVAVIWYDGSKKYY | NO: |
| | | | IPARFSGSGSGTDFTLTIS | 64 | VDSVKGRFTISRDNSKNTLY | 65 |
| | | | SLEPEDFAVYYCQQRSNWP | | LQMNSLRAEDTAVYYCARQM | |
| | | | PLTFGGGTKVEIK | | GYWHFDLWGRGTLVTVSS | |
| 27H5- | human | — | EIVLTQSPRTLSLSPGERA | SEQ | QVQLVESGGGVVQPGRSLRL | SEQ |
| VL1 | CD3 | | TLSCRASQSVSSSYLAWYQ | ID | SCAASGFTFRSYGMHWVRQA | ID |
| | | | QKPGQAPRLLIYGASSRAT | NO: | PGKGLEWVAIIWYDGSKKNY | NO: |
| | | | GIPDRFSGSGSGTDFTLTI | 66 | ADSVKGRFTISRDNSKNTLY | 67 |
| | | | SRLDPEDFAVYYCQQYGSS | | LQMNSLRAEDTAVYYCARGT | |
| | | | PITFGQGTRLEIK | | GYNWFDPWGQGTLVTVSS | |
| 23F10 | human | — | EIVLTQSPATLSLSPGERA | SEQ | QVQLVQSGGGVVQSGRSLRL | SEQ |
| | CD3 | | TLSCRASQSVSSYLAWYQQ | ID | SCAASGFKFSGYGMHWVRQA | ID |
| | | | KPGQAPRLLIYDASNRATG | NO: | PGKGLEWVAVIWYDGSKKYY | NO: |
| | | | IPARFSGSGSGTDFTLTIS | 68 | VDSVKGRFTISRDNSKNTLY | 69 |
| | | | SLEPEDFAVYYCQQRSNWP | | LQMNSLRGEDTAVYYCARQM | |
| | | | PLTFGGGTKVEIK | | GYWHFDLWGRGTLVTVSS | |
| 15C3- | human | — | EIVLTQSPATLSLSPGERA | SEQ | QVQLVQSGGGVVQPGRSLRL | SEQ |
| VL1 | CD3 | | TLSCRASQSVSSYLAWYQQ | ID | SCVASGFTFSSYGMHWVRQA | ID |
| | | | KPGQAPRLLIYDASNRATG | NO: | PGKGLEWVAAIWYNGRKQDY | NO: |
| | | | IPARFSGSGSGTDFTLTIS | 70 | ADSVKGRFTISRDNSKNTLY | 71 |
| | | | SLEPEDFAVYYCQQRSNWP | | LQMNSLRAEDTAVYYCTRGT | |
| | | | WTFGQGTKVEIK | | GYNWFDPWGQGTLVTVSS | |
| 15C3- | human | — | AIQLTQSPSSLSASVGDRV | SEQ | QVQLVQSGGGVVQPGRSLRL | SEQ |
| VL2 | CD3 | | TITCRASQGISSALAWYQQ | ID | SCVASGFTFSSYGMHWVRQA | ID |
| | | | KPGKAPKLLIYDASSLESG | NO: | PGKGLEWVAAIWYNGRKQDY | NO: |
| | | | VPSRFSGSGSGTDFTLTIS | 72 | ADSVKGRFTISRDNSKNTLY | 73 |
| | | | SLQPEDFATYYCQQFNSYP | | LQMNSLRAEDTAVYYCTRGT | |
| | | | ITFGQGTRLEIK | | GYNWFDPWGQGTLVTVSS | |
| hu12F6 | human | — | QIVLSQSPAILSASPGEKV | SEQ | QVQLQQSGAELARPGASVKM | SEQ |
| | CD3 | | TMTCRASSSVSYMHWYQQK | ID | SCKASGYTFTSYTMHWVKQR | ID |
| | | | PGSSPKPWIYATSNLASGV | NO: | PGQGLEWIGYINPSSGYTKY | NO: |
| | | | PARFSGSGSGTSYSLTISR | 74 | NQKFKDKATLTADKSSSTAY | 75 |
| | | | VEAEDAATYYCQQWSSNPP | | MQLSSLTSEDSAVYYCARWQ | |
| | | | TFGGGTKLETKR | | DYDVYFDYWGQGTTLTVSS | |

TABLE 4
Anti-human CD16a scFv sequences

| Clone Name and | |
|---|---|
| SEQ ID NO: | Clone scFv Sequence |
| SEQ ID NO: 76 | DIVLTQSPASLAVSLGQRATISCKASQSVDEDGDSFMNWYQQKPGQPPKLLIYTTS |
| 3G8 | NLESGIPARFSASGSGTDFTLNIHPVEEEDTATYYCQQSNEDPYTFGGGTKLELKG |
| | GGGSGGGGSGGGGSQVTLKESGPGILQPSQTLSLTCSFSGFSLRTSGMGVGWIRQP |
| | SGKGLEWLAHIWWDDDKRYNPALKSRLTISKDTSSNQVFLKIASVDTADTATYYCA |
| | QINPAWFAYWGQGTLVTVSA |
| SEQ ID NO: 77 | DIVLTQSPDSLAVSLGERATINCKASQSVDEDGDSFMNWYQQKPGQPPKLLIYTTS |
| Humanized | NLESGVPDRFSGSGSGTDFTLTIRPLQAEDVAVYYCQQSNEDPYTFGQGTKLEIKG |
| 3G8 clone | GGGSGGGGSGGGGSQVTLKESGPALVKPTQTLTLTCTFSGFSLSTSGMGVGWIRQP |
| Mu 3 | PGKALEWLAHIWWDDDKRYNPALKSRLTISKDTSKNQVVLTMTNMDPVDTATYYCA |
| | QINPAWFAYWGQGTLVTVSS |
| SEQ ID NO: 78 | DIELTQESALTTSPGETVTLTCRSNTGTVTTSNYANWVQEKPDHLFTGLIGHTNNR |
| muA9 | APGVPARFSGSLIGDKAALTITGAQTEDEAIYFCALWYNNHWVEGGGTKLEIKGGG |
| | GSGGGGSGGGGSEVQLQESGAELVRPGTSVKISCKASGYTFTNYWLGWVKQRPGHG |
| | LEWIGDIYPGGGYTNYNEKFKGKATVTADTSSRTAYVQVRSLTSEDSAVYFCARSA |
| | SWYFDVWGQGTTVTVSS |
| SEQ ID NO: 79 | DIELTQSPSSLSASVGDRVTITCRSNTGTVTTSNYASWYQQKPGKAPGGLIGHTNN |
| huA9 | RAPGVPSRFSGSLSGADATLTISSLKPEDLATYYCALWYNHWVFGQGTKLEIKGGG |
| | GSGGGGSGGGGSEVQLQESGGGLVQPGDSLRLSCAASGFTFSNYWLGWIRQAPGKG |
| | PEWVGDIYPGGGYTNYNEKFKGRFTISADTSKNIAYLQVNSLRAEDTAVYYCARSA |
| | SWYFDVWGQGTLVTVSS |
| SEQ ID NO: 80 | SYVLTQPSSVSVAPGQTATISCGGHNIGSKNVHWYQQRPGQSPVLVIYQDNKRPSG |
| LSIV21 | IPERFSGSNSGNTATLTISGTQAMDEADYYCQVWDNYSVLFGGGTKLTVLGGGGSG |
| | GGGSGGGGSQVQLVQSGAEVKKPGESLKVSCKASGYTFTSYYMHWVRQAPGQGLEW |
| | MGIINPSGGSTSYAQKFQGRVTMTRDTSTSTVYMELSSLRSEDTAVYYCARGSAYY |
| | YDFADYWGQGTLVTVSS |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A molecule comprising a first antigen-binding domain comprising:
(i) an scFv light chain CDR1 comprising or consisting of SEQ ID NO: 8;
(ii) an scFv light chain CDR2 comprising or consisting of SEQ ID NO: 9 or SEQ ID NO: 23;
(iii) an scFv light chain CDR3 comprising or consisting of SEQ ID NO: 10;
(iv) an scFV heavy chain CDR1 comprising or consisting of SEQ ID NO: 17;
(v) an scFV heavy chain CDR2 comprising or consisting of SEQ ID NO: 18 or SEQ ID NO: 27; and
(vi) an scFV heavy chain CDR3 comprising or consisting of SEQ ID NO: 19.

2. The molecule of claim 1, wherein the first antigen-binding domain comprises:
(a) the scFv light chain CDR2 comprising or consisting of SEQ ID NO: 23; or
(b) the scFv heavy chain CDR2 comprising or consisting of SEQ ID NO: 27; or
(c) the scFv light chain CDR2 comprising or consisting of SEQ ID NO: 23 and the scFv heavy chain CDR2 comprising or consisting of SEQ ID NO: 27.

3. The molecule of claim 1 or 2, wherein the first antigen-binding domain comprises:
(i) an scFv light chain comprising a sequence at least 90% identical to SEQ ID NO: 7 or SEQ ID NO: 22; and
(ii) an scFv heavy chain comprising a sequence at least 90% identical to SEQ ID NO: 16 or SEQ ID NO: 26.

4. The molecule of claim 3, wherein the first antigen-binding domain comprises:
(a) the scFv light chain comprising the sequence at least 90% identical to SEQ ID NO: 7 and the scFv heavy chain comprising a sequence at least 90% identical to SEQ ID NO: 26; or
(b) the scFv light chain comprising a sequence at least 90% identical to SEQ ID NO: 22 and the scFv heavy chain comprising a sequence at least 90% identical to SEQ ID NO: 16; or
(c) the scFv light chain comprising a sequence at least 90% identical to SEQ ID NO: 22 and the scFv heavy chain comprising a sequence at least 90% identical to SEQ ID NO: 26.

5. The molecule of any one of claims 1-4, wherein the first antigen-binding domain comprises:
(i) an scFv light chain comprising a sequence at least 98% identical to SEQ ID NO: 7 or SEQ ID NO: 22; and
(ii) an scFv heavy chain comprising a sequence at least 98% identical to SEQ ID NO: 16 or SEQ ID NO: 26.

6. The molecule of claim 5, wherein the first antigen-binding domain comprises:
(a) the scFv light chain comprising the sequence at least 98% identical to SEQ ID NO: 7 and the scFv heavy chain comprising a sequence at least 98% identical to SEQ ID NO: 26; or
(b) the scFv light chain comprising a sequence at least 98% identical to SEQ ID NO: 22 and the scFv heavy chain comprising a sequence at least 98% identical to SEQ ID NO: 16; or
(c) the scFv light chain comprising a sequence at least 98% identical to SEQ ID NO: 22 and the scFv heavy chain comprising a sequence at least 98% identical to SEQ ID NO: 26.

7. The molecule of any one of claims 1-6, wherein the first antigen-binding domain comprises:
(i) an scFv light chain comprising or consisting of SEQ ID NO: 7 or SEQ ID NO: 22; and
(ii) an scFv heavy chain comprising or consisting of SEQ ID NO: 16 or SEQ ID NO: 26.

8. The molecule of claim 7, wherein the first antigen-binding domain comprises:
(a) the scFv light chain comprising or consisting of SEQ ID NO: 7 and the scFv heavy chain comprising or consisting of SEQ ID NO: 26; or
(b) the scFv light chain comprising or consisting of SEQ ID NO: 22 and the scFv heavy chain comprising or consisting of SEQ ID NO: 16; or
(c) the scFv light chain comprising or consisting of SEQ ID NO: 22 and the scFv heavy chain comprising or consisting of SEQ ID NO: 26.

9. The molecule of any one of claims 1-8, wherein the molecule is selected from the group consisting of an antibody, an antibody fragment, a single chain variable fragment (scFv), a chimeric antigen receptor (CAR), a T cell receptor (TCR), a TCR mimic, a tandem scFv, a bispecific T cell engager, a diabody, a single-chain diabody (scDb), an scFv-Fc, a bispecific antibody, and a dual-affinity re-targeting antibody (DART).

10. The molecule of any one of claims 1-9, wherein the molecule further comprises a second antigen-binding domain that can bind to an effector cell receptor selected from the group consisting of CD3, CD28, CD4, CD8, CD16a, NKG2D, PD-1, CTLA-4, 4-1BB, OX40, ICOS, and CD27.

11. The molecule of claim 10, wherein the second antigen-binding domain can bind to CD3.

12. The molecule of claim 11, wherein the second antigen-binding domain that can bind to CD3 comprises a variable light chain and a variable heavy chain selected from those shown in Table 3.

13. The molecule of claim 12, wherein the second antigen-binding domain that can bind to CD3 comprises or consists of any one of those shown in Table 2 (SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51).

14. The molecule of any one of claims 1-13, wherein the molecule is a single-chain diabody (scDb).

15. The molecule of claim 14, wherein the single-chain diabody comprises, in order from N- to C-terminus:
(i) an scFv light chain comprising:
(a) an scFv light chain CDR1 comprising or consisting of SEQ ID NO: 8;
(b) an scFv light chain CDR2 comprising or consisting of SEQ ID NO: 9 or SEQ ID NO: 23;
(c) an scFv light chain CDR3 comprising or consisting of SEQ ID NO: 10
(ii) an antigen binding domain that can bind to an effector cell receptor selected from the group consisting of CD3, CD28, CD4, CD8, CD16a, NKG2D, PD-1, CTLA-4, 4-1BB, OX40, ICOS, and CD27; and
(iii) an scFv heavy chain comprising:
(a) an scFV heavy chain CDR1 comprising or consisting of SEQ ID NO: 17;
(b) an scFV heavy chain CDR2 comprising or consisting of SEQ ID NO: 18 or SEQ ID NO: 27; and
(c) an scFV heavy chain CDR3 comprising or consisting of SEQ ID NO: 19.

16. The molecule of claim 15, wherein the single-chain diabody comprises:
(a) the scFv light chain CDR2 comprising or consisting of SEQ ID NO: 23; or
(b) the scFv heavy chain CDR2 comprising or consisting of SEQ ID NO: 27; or
(c) the scFv light chain CDR2 comprising or consisting of SEQ ID NO: 23 and the scFv heavy chain CDR2 comprising or consisting of SEQ ID NO: 27.

17. The molecule of any one of claims 14-16, wherein the single-chain diabody comprises, in order from N- to C-terminus:
(i) an scFv light chain comprising or consisting of SEQ ID NO: 7 or SEQ ID NO: 22;
(ii) an antigen binding domain that can bind to an effector cell receptor selected from the group consisting of CD3, CD28, CD4, CD8, CD16a, NKG2D, PD-1, CTLA-4, 4-1BB, OX40, ICOS, and CD27; and
(iii) an scFv heavy chain comprising or consisting of SEQ ID NO: 16 or SEQ ID NO: 26.

18. The molecule of claim 17, wherein the single-chain diabody comprises:
(a) the scFv light chain comprising or consisting of SEQ ID NO: 7 and the scFv heavy chain comprising or consisting of SEQ ID NO: 26; or
(b) the scFv light chain comprising or consisting of SEQ ID NO: 22 and the scFv heavy chain comprising or consisting of SEQ ID NO: 16; or
(c) the scFv light chain comprising or consisting of SEQ ID NO: 22 and the scFv heavy chain comprising or consisting of SEQ ID NO: 26.

19. The molecule of claim 15 or claim 17, wherein the antigen binding domain is a CD3 antigen binding domain.

20. The molecule of claim 19, wherein the CD3 antigen binding domain comprises a variable light chain and a variable heavy chain selected from those shown in Table 3.

21. The molecule of claim 20, wherein the variable light chain and variable heavy chain of the antigen binding domain are separated by a linker, preferably a 3×G₄S linker (SEQ ID NO: 13).

22. The molecule of any one of claims 19-21, wherein the antigen-binding domain that can bind to CD3 comprises or consists of any one of those shown in Table 2 (SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51).

23. The molecule of any one of claims 15-22, further comprising a first linker between the scFv light chain and the antigen binding domain and a second linker between the antigen binding domain and the scFv heavy chain.

24. The molecule of claim 23, wherein the first linker comprises or consists of G4S (SEQ ID NO: 11) and the second linker comprises or consists of G4S (SEQ ID NO: 15).

25. A method for treating a mammal having a cancer expressing a mutant peptide comprising or consisting of HMTEVVRHC (SEQ ID NO: 1), the method comprising:
administering to the mammal a molecule of any one of claims 1 to 24.

26. The method of claim 25, wherein said mammal is a human.

27. The method of claim 25 or claim 26, wherein said cancer is Hodgkin's lymphoma, non-Hodgkin's lymphoma, acute myeloid leukemia, acute lymphoblastic leukemia, multiple myeloma, a myelodysplastic syndrome (MDS), a myeloproliferative disease, lung cancer, pancreatic cancer, gastric cancer, colorectal cancer, ovarian cancer, endometrial cancer, biliary tract cancer, liver cancer, breast cancer, prostate cancer, esophageal cancer, stomach cancer, kidney cancer, bone cancer, soft tissue cancer, head and neck cancer, glioblastoma multiforme, astrocytoma, thyroid cancer, germ cell tumor, or melanoma.


SEQUENCE LISTING (80 sequences)

SEQ ID NO: 1 - protein, 9 aa, synthetic construct
HMTEVVRHC

SEQ ID NO: 2 - protein, 9 aa, Homo sapiens
HMTEVVRRC

SEQ ID NO: 3 - protein, 393 aa, Homo sapiens [sequence omitted]

SEQ ID NO: 4 - protein, 245 aa, synthetic construct
DIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKRTGGGSGGGGSGGGASEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSS

SEQ ID NO: 5 - protein, 504 aa, synthetic construct
MYRMQLLSCIALSLALVTNSDIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSSHHHHHH

SEQ ID NO: 6 - protein, 20 aa, synthetic construct
MYRMQLLSCIALSLALVTNS

SEQ ID NO: 7 - protein, 108 aa, synthetic construct
DIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIK

SEQ ID NO: 8 - protein, 11 aa, synthetic construct
RASQDVNTAVA

SEQ ID NO: 9 - protein, 7 aa, synthetic construct
SAYFLYS

SEQ ID NO: 10 - protein, 9 aa, synthetic construct
QQYSRYSPV

SEQ ID NO: 11 - protein, 5 aa, synthetic construct
GGGGS

SEQ ID NO: 12 - protein, 122 aa, synthetic construct
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSS

SEQ ID NO: 13 - protein, 15 aa, synthetic construct
GGGGSGGGGSGGGGS

SEQ ID NO: 14 - protein, 107 aa, synthetic construct
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 15 - protein, 5 aa, synthetic construct
GGGGS

SEQ ID NO: 16 - protein, 121 aa, synthetic construct
EVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSS

SEQ ID NO: 17 - protein, 9 aa, synthetic construct
FNVYASGMH

SEQ ID NO: 18 - protein, 13 aa, synthetic construct
VAKIYPDSDYTYY

SEQ ID NO: 19 - protein, 12 aa, synthetic construct
SRDSSFYYVYAM

SEQ ID NO: 20 - protein, 245 aa, synthetic construct
DIQMTQSPSSLSASVGDRVTITCRASQDVNTAVAWYQQKPGKAPKLLIYSAYSLYSGVPSRFSGSRSGTDFTLTISSLQPEDFATYYCQQYSRYSPVTFGQGTKVEIKRTGGGSGGGGSGGGASEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSS

SEQ ID NO: 21 - protein, 503 aa, synthetic construct
MYRMQLLSCIALSLALVTNSDIQMTQSPSSLSASVGDRVTITCRASQDVNTAVAWYQQKPGKAPKLLIYSAYSLYSGVPSRFSGSRSGTDFTLTISSLQPEDFATYYCQQYSRYSPVTFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFNVYASGMHWVRQAPGKGLEWVAKIYPDSDYTYYADSVKGRFTISADTSKNTAYLQMNSLRAEDTAVYYCSRDSSFYYVYAMDYWGQGTLVTVSS

SEQ ID NO: 22 - protein, 108 aa, synthetic construct
DIQMTQSPSSLSASVGDRVTITCRASQDVNTAVAWYQQKPGKAPKLLIYSAYSLYSGVPSRFSGSRSGTDFTLTISSLQPEDFATYYCQQYSRYSPVTFGQGTKVEIK

SEQ ID NO: 23 - protein, 7 aa, synthetic construct
SAYSLYS

SEQ ID NO: 24 - protein, 245 aa, synthetic construct
DIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKRTGGGSGGGGSGGGASEVQLVESGGGLVQPGGSLRLSCAASGFNVYASGMHWVRQAPGKGLEWVAKIYPDSDITYYADSVKGRFTISADTSKNTAYLQMNSLRAEDTAVYYCSRDSSFYYVYAMDYWGQGTLVTVSS

SEQ ID NO: 25 - protein, 503 aa, synthetic construct
MYRMQLLSCIALSLALVTNSDIQMTQSPSSLSASVGDRVTITCRASQDVNTAVAWYQQKPGKAPKLLIYSAYFLYSGVPSRFSGSRSGTDFTLTISSLQPEDFATYYCQQYSRYSPVTFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFNVYASGMHWVRQAPGKGLEWVAKIYPDSDITYYADSVKGRFTISADTSKNTAYLQMNSLRAEDTAVYYCSRDSSFYYVYAMDYWGQGTLVTVSS

SEQ ID NO: 26 - protein, 121 aa, synthetic construct
EVQLVESGGGLVQPGGSLRLSCAASGFNVYASGMHWVRQAPGKGLEWVAKIYPDSDITYYADSVKGRFTISADTSKNTAYLQMNSLRAEDTAVYYCSRDSSFYYVYAMDYWGQGTLVTVSS

SEQ ID NO: 27 - protein, 13 aa, synthetic construct
VAKIYPDSDITYY

SEQ ID NO: 28 - protein, 244 aa, synthetic construct
DIQMTQSPSSLSASVGDRVTITCRASQDVNTAVAWYQQKPGKAPKLLIYSAYSLYSGVPSRFSGSRSGTDFTLTISSLQPEDFATYYCQQYSRYSPVTFGQGTKVEIKRTGGGSGGGGSGGGASEVQLVESGGGLVQPGGSLRLSCAASGFNVYASGMHWVRQAPGKGLEWVAKIYPDSDITYYADSVKGRFTISADTSKNTAYLQMNSLRAEDTAVYYCSRDSSFYYVYAMDYWGQGTLVTVS

SEQ ID NO: 29 - protein, 503 aa, synthetic construct
MYRMQLLSCIALSLALVTNSDIQMTQSPSSLSASVGDRVTITCRASQDVNTAVAWYQQKPGKAPKLLIYSAYSLYSGVPSRFSGSRSGTDFTLTISSLQPEDFATYYCQQYSRYSPVTFGQGTKVEIKGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSEVQLVESGGGLVQPGGSLRLSCAASGFNVYASGMHWVRQAPGKGLEWVAKIYPDSDITYYADSVKGRFTISADTSKNTAYLQMNSLRAEDTAVYYCSRDSSFYYVYAMDYWGQGTLVTVSS

SEQ ID NO: 30 - protein, 108 aa, synthetic construct; identical to SEQ ID NO: 7

SEQ ID NO: 31 - protein, 121 aa, synthetic construct; identical to SEQ ID NO: 16

SEQ ID NO: 32 - protein, 108 aa, synthetic construct; identical to SEQ ID NO: 22

SEQ ID NO: 33 - protein, 121 aa, synthetic construct; identical to SEQ ID NO: 16

SEQ ID NO: 34 - protein, 108 aa, synthetic construct; identical to SEQ ID NO: 7

SEQ ID NO: 35 - protein, 121 aa, synthetic construct; identical to SEQ ID NO: 26

SEQ ID NO: 36 - protein, 108 aa, synthetic construct; identical to SEQ ID NO: 22

SEQ ID NO: 37 - protein, 121 aa, synthetic construct; identical to SEQ ID NO: 26

SEQ ID NO: 38 - protein, 244 aa, synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 39 - protein, 244 aa, synthetic construct
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSS

SEQ ID NO: 40 - protein, 240 aa, synthetic construct
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKGGGGSGGGGSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSS

SEQ ID NO: 41 - protein, 249 aa, synthetic construct
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGSGGGGSEVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 42 - protein, 240 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 43 - protein, 240 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINGGGGSGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 44 - protein, 249 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 45 - protein, 241 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGGGGSGGGGSGGGGSQVQLVESGGGVVQPGRSLRLSCAASGFKFSGYGMHWVRQAPGKGLEWVAVIWYDGSKKYYVDSVKGRFTISRDNSKNTLYLQMNSLRAEDTAVYYCARQMGYWHFDLWGRGTLVTVSS

SEQ ID NO: 46 - protein, 241 aa, synthetic construct
EIVLTQSPRTLSLSPGERATLSCRASQSVSSSYLAWYQQKPGQAPRLLIYGASSRATGIPDRFSGSGSGTDFTLTISRLDPEDFAVYYCQQYGSSPITFGQGTRLEIKGGGGSGGGGSGGGGSQVQLVESGGGVVQPGRSLRLSCAASGFTFRSYGMHWVRQAPGKGLEWVAIIWYDGSKKNYADSVKGRFTISRDNSKNTLYLQMNSLRAEDTAVYYCARGTGYNWFDPWGQGTLVTVSS

SEQ ID NO: 47 - protein, 239 aa, synthetic construct
DILMTQSPSSLSASVGDRVTITCRASQGISSALAWYQQKPGKAPKLLIYYASSLQSGVPSRFSGSGSGTDYTLTISSLQPEDFATYYCQQYYSTLTFGGGTKVEIKGGGGSGGGGSGGGGSQVQLVESGGGVVQPGRSLRLSCAASGFTFRSYGMHWVRQAPGKGLEWVAIIWYDGSKKNYADSVKGRFTISRDNSKNTLYLQMNSLRAEDTAVYYCARGTGYNWFDPWGQGTLVTVSS

SEQ ID NO: 48 - protein, 241 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIKGGGGSGGGGSGGGGSQVQLVQSGGGVVQSGRSLRLSCAASGFKFSGYGMHWVRQAPGKGLEWVAVIWYDGSKKYYVDSVKGRFTISRDNSKNTLYLQMNSLRGEDTAVYYCARQMGYWHFDLWGRGTLVTVSS

SEQ ID NO: 49 - protein, 240 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKGGGGSGGGGSGGGGSQVQLVQSGGGVVQPGRSLRLSCVASGFTFSSYGMHWVRQAPGKGLEWVAAIWYNGRKQDYADSVKGRFTISRDNSKNTLYLQMNSLRAEDTAVYYCTRGTGYNWFDPWGQGTLVTVSS

SEQ ID NO: 50 - protein, 240 aa, synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPITFGQGTRLEIKGGGGSGGGGSGGGGSQVQLVQSGGGVVQPGRSLRLSCVASGFTFSSYGMHWVRQAPGKGLEWVAAIWYNGRKQDYADSVKGRFTISRDNSKNTLYLQMNSLRAEDTAVYYCTRGTGYNWFDPWGQGTLVTVSS

SEQ ID NO: 51 - protein, 241 aa, synthetic construct
QIVLSQSPAILSASPGEKVTMTCRASSSVSYMHWYQQKPGSSPKPWIYATSNLASGVPARFSGSGSGTSYSLTISRVEAEDAATYYCQQWSSNPPTFGGGTKLETKRGGGGSGGGGSGGGGSQVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSS

SEQ ID NO: 52 - protein, 107 aa, synthetic construct; identical to SEQ ID NO: 14

SEQ ID NO: 53 - protein, 122 aa, synthetic construct; identical to SEQ ID NO: 12

SEQ ID NO: 54 - protein, 107 aa, synthetic construct [sequence omitted]

SEQ ID NO: 55 - protein, 122 aa, synthetic construct [sequence omitted]

SEQ ID NO: 56 - protein, 106 aa, synthetic construct
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 57 - protein, 119 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSS

SEQ ID NO: 58 - protein, 109 aa, synthetic construct
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVL

SEQ ID NO: 59 - protein, 125 aa, synthetic construct
EVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 60 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 61 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 62 - protein, 106 aa, synthetic construct [sequence omitted]

SEQ ID NO: 63 - protein, 119 aa, synthetic construct [sequence omitted]

SEQ ID NO: 64 - protein, 108 aa, synthetic construct [sequence omitted]

SEQ ID NO: 65 - protein, 118 aa, synthetic construct [sequence omitted]

SEQ ID NO: 66 - protein, 108 aa, synthetic construct
EIVLTQSPRTLSLSPGERATLSCRASQSVSSSYLAWYQQKPGQAPRLLIYGASSRATGIPDRFSGSGSGTDFTLTISRLDPEDFAVYYCQQYGSSPITFGQGTRLEIK

SEQ ID NO: 67 - protein, 118 aa, synthetic construct
QVQLVESGGGVVQPGRSLRLSCAASGFTFRSYGMHWVRQAPGKGLEWVAIIWYDGSKKNYADSVKGRFTISRDNSKNTLYLQMNSLRAEDTAVYYCARGTGYNWFDPWGQGTLVTVSS

SEQ ID NO: 68 - protein, 108 aa, synthetic construct [sequence omitted]

SEQ ID NO: 69 - protein, 118 aa, synthetic construct
QVQLVQSGGGVVQSGRSLRLSCAASGFKFSGYGMHWVRQAPGKGLEWVAVIWYDGSKKYYVDSVKGRFTISRDNSKNTLYLQMNSLRGEDTAVYYCARQMGYWHFDLWGRGTLVTVSS

SEQ ID NO: 70 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 71 - protein, 118 aa, synthetic construct
QVQLVQSGGGVVQPGRSLRLSCVASGFTFSSYGMHWVRQAPGKGLEWVAAIWYNGRKQDYADSVKGRFTISRDNSKNTLYLQMNSLRAEDTAVYYCTRGTGYNWFDPWGQGTLVTVSS

SEQ ID NO: 72 - protein, 107 aa, synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPITFGQGTRLEIK

SEQ ID NO: 73 - protein, 118 aa, synthetic construct; identical to SEQ ID NO: 71

SEQ ID NO: 74 - protein, 107 aa, synthetic construct
QIVLSQSPAILSASPGEKVTMTCRASSSVSYMHWYQQKPGSSPKPWIYATSNLASGVPARFSGSGSGTSYSLTISRVEAEDAATYYCQQWSSNPPTFGGGTKLETKR

SEQ ID NO: 75 - protein, 119 aa, synthetic construct
QVQLQQSGAELARPGASVKMSCKASGYTFTSYTMHWVKQRPGQGLEWIGYINPSSGYTKYNQKFKDKATLTADKSSSTAYMQLSSLTSEDSAVYYCARWQDYDVYFDYWGQGTTLTVSS

SEQ ID NO: 76 - protein, 244 aa, synthetic construct
DIVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLELKGGGGSGGGGSGGGGSQVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSA

SEQ ID NO: 77 - protein, 244 aa, synthetic construct
DIVLTQSPDSLAVSLGERATINCKASQSVDFDGDSFMNWYQQKPGQPPKLLIYTTSNLESGVPDRFSGSGSGTDFTLTIRPLQAEDVAVYYCQQSNEDPYTFGQGTKLEIKGGGGSGGGGSGGGGSQVTLKESGPALVKPTQTLTLTCTFSGFSLSTSGMGVGWIRQPPGKALEWLAHIWWDDDKRYNPALKSRLTISKDTSKNQVVLTMTNMDPVDTATYYCAQINPAWFAYWGQGTLVTVSS

SEQ ID NO: 78 - protein, 241 aa, synthetic construct
DIELTQESALTTSPGETVTLTCRSNTGTVTTSNYANWVQEKPDHLFTGLIGHTNNRAPGVPARFSGSLIGDKAALTITGAQTEDEAIYFCALWYNNHWVFGGGTKLEIKGGGGSGGGGSGGGGSEVQLQESGAELVRPGTSVKISCKASGYTFTNYWLGWVKQRPGHGLEWIGDIYPGGGYTNYNEKFKGKATVTADTSSRTAYVQVRSLTSEDSAVYFCARSASWYFDVWGQGTTVTVSS

SEQ ID NO: 79 - protein, 241 aa, synthetic construct
DIELTQSPSSLSASVGDRVTITCRSNTGTVTTSNYASWYQQKPGKAPGGLIGHTNNRAPGVPSRFSGSLSGADATLTISSLKPEDLATYYCALWYNHWVFGQGTKLEIKGGGGSGGGGSGGGGSEVQLQESGGGLVQPGDSLRLSCAASGFTFSNYWLGWIRQAPGKGPEWVGDIYPGGGYTNYNEKFKGRFTISADTSKNIAYLQVNSLRAEDTAVYYCARSASWYFDVWGQGTLVTVSS

SEQ ID NO: 80 - protein, 241 aa, synthetic construct
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVLGGGGSGGGGSGGGGSQVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSS